(19) United States
(12) Patent Application Publication
(10) Pub. No.: US 2020/0032326 A1
(43) Pub. Date: Jan. 30, 2020

(54) ULTRASENSITIVE ASSAYS FOR DETECTION OF SHORT NUCLEIC ACIDS

(71) Applicant: Trustees of Tufts College, Medford, MA (US)

(72) Inventors: David R. Walt, Boston, MA (US); Limor Cohen, Boston, MA (US); Mark Hartman, Arlington, MA (US)

(73) Assignee: Trustees of Tufts College, Medford, MA (US)

(21) Appl. No.: 16/424,989

(22) Filed: May 29, 2019

Related U.S. Application Data
(60) Provisional application No. 62/677,618, filed on May 29, 2018.

(51) Int. Cl. C12Q 1/6832; C12Q 1/6876

(52) U.S. Cl. CPC C12Q 1/6832; C12Q 2600/178; C12Q 1/6876

(57) ABSTRACT

Described herein are ultrasensitive methods to detect the presence and/or measure the levels of short target nucleic acids, such as microRNAs, in a sample. Such a method can involve the use of a capture probe and a detection probe, each of which is complementary to a segment of the short target nucleic acid. The capture probe and a detection probe may be hybridized with the target nucleic acid in the sample and the complex thus formed can be detected, for example, by a single molecular array assay.

### CROSS REFERENCE TO RELATED APPLICATIONS

The present application claims priority under 35 U.S.C. § 119(e) to U.S. Provisional Application Ser. No. 62/677,618, filed May 29, 2018, which is incorporated herein by reference.

### GOVERNMENT SUPPORT

This invention was made with government support under Grant No. BC100510/W81XWH-11-1-0814, awarded by the United States Department of Defense, and Grant No. HR0011-12-2-0001, awarded by the Defense Advanced Research Projects Agency (DARPA). The Government has certain rights in this invention.

### BACKGROUND OF THE INVENTION

MicroRNAs represent a class of small non-coding regulatory RNAs that play a major role in the control of gene expression by repressing protein synthesis at the post-transcriptional level. As key components of gene expression regulation, microRNAs are involved in virtually every biological and thus represent a very rich source of biological information. Specific microRNAs such as miR-21, miR-141, and the let-7 family, have demonstrated associations with various types of cancers.

Ultrasensitive detection of single molecules of microRNA is traditionally challenging to achieve via conventional detection methods mainly due to their small size, frequent sequence similarity among different microRNAs, lack of tissue-specific expression, and low abundance. Currently, qRT-PCR is the gold standard for nucleic acid detection due to its high sensitivity in comparison to microarray techniques. However, the short length of microRNA makes it incompatible with standard PCR primers. Further, detection of multiple microRNAs with microarray techniques requires extensive pre-amplification to achieve adequate sensitivity. The northern blot technique has been widely used to detect microRNAs with high specificity. However, this technique is quite time-consuming and has low sensitivity. Available methods for microRNA detection require specialized skills and materials and also involve pre-amplification of the target microRNA to achieve adequate sensitivity.

Accordingly, there is a need to develop new assays for detecting microRNAs and other nucleic acids with high sensitivity and specificity.

### SUMMARY OF THE INVENTION

The present disclosure is based on the development of an ultrasensitive assay method for detecting nucleic acids such as microRNAs with high sensitivity and specificity.

Accordingly, one aspect of the present disclosure provides a method for detecting a target nucleic acid in a sample, comprising: (a) providing a sample suspected of containing a first target nucleic acid, wherein the first target nucleic acid is about 15-50-nucleotides in length, (b) incubating the sample with a first capture probe and a first detection probe to form a first complex of the first target nucleic acid, the first capture probe, and the first detection probe; wherein the first capture probe is immobilized on a first support member and the first detection probe is conjugated to a first labeling agent, (c) washing the first complex to remove unbound first capture and first detection probes, (d) measuring a first signal released, directly or indirectly, from the first labeling agent in the first complex, and (e) determining presence or a level of the first target nucleic acid in the sample based on the intensity of the first signal obtained in step (d). The first capture probe and the first detection probe each comprise a nucleotide sequence that is complementary to a first segment of the first target nucleic acid and a second segment of the first target nucleic acid, respectively. Further, the first segment and second segment of the first target nucleic acid do not overlap.

In some embodiments, the first target nucleic acid is about 18-25-nucleotides in length. In some instances, the first target nucleic acid is RNA, e.g., a microRNA. In one example, the first target nucleic acid is mature microRNA. In some embodiments, the first segment and the second segment of the first target nucleic acid differ in length by less than or equal to about 5 nucleotides. In some embodiments, the first target nucleic acid is not enriched or amplified prior to step (a).

The sample suspected of containing a first target nucleic acid can be a biological sample, which may be obtained from a human subject. In one example, the sample is serum (e.g., serum obtained from a human subject).

In some embodiments, the first capture probe, the first detection probe, or both comprise one or more locked nucleic acids (LNAs). In some embodiments, the first capture probe and the first detection probe have low cross-reactivity in the absence of the first target nucleic acid. In some instances, the first capture probe and the first detection probe collectively are complementary to the whole length of the first target nucleic acid. In some examples, the first capture probe, the first detection probe, or both have a melting temperature ranging from about 30° C. and about 90° C. In some cases, the melting temperature of the first capture probe differs from that of the first detection probe by up to about 40° C. In some embodiments, the first support member to which the first capture probe is attached is a magnetic bead. In some embodiments, the first labeling agent conjugated to the first detection probe is biotin.

In some embodiments, the sample may be suspected of containing a second target nucleic acid, which is about 15-50-nucleotides in length. In some such embodiments, in step (b), the sample is further incubated with a second capture probe, and a second detection probe to form a second complex of the second target nucleic acid, the second capture probe, and the second detection probe, the second capture probe being immobilized on a second support member and comprising a nucleotide sequence complementary to a first segment of the second nucleic acid and the second detection probe being conjugated to a second labeling agent and comprising a nucleotide sequence complementary to a second segment of the second target nucleic acid, which does not overlap with the first segment, and wherein the method further comprises measuring a second signal released, directly or indirectly, from the second labeling agent in the second complex, and determining presence or a level of the second target nucleic acid in the sample based on the intensity of the second signal.

The sample may be suspected of containing a third target nucleic acid, which is about 15-50-nucleotides in length. In some such embodiments, in step (b), the sample is further incubated with a third capture probe, and a third detection probe to form a third complex of the third target nucleic acid, the third capture probe, and the third detection probe, the third capture probe being immobilized on a third support member and comprising a nucleotide sequence complementary to a first segment of the third nucleic acid and the third detection probe being conjugated to a third labeling agent and comprising a nucleotide sequence complementary to a second segment of the third target nucleic acid, which does not overlap with the first segment, and wherein the method further comprises measuring a third signal released, directly or indirectly, from the third labeling agent in the third complex, and determining presence or a level of the third target nucleic acid in the sample based on the intensity of the third signal.

In some embodiments, the second target nucleic acid, the third target nucleic acid, or both are about 18-25-nucleotides in length. The second target nucleic acid, the third nucleic acid, or both may be RNA molecules. For example, the RNA molecules are mature microRNAs.

In some examples, the first support member, the second support member, and the third support member are paramagnetic beads. The first support member, the second support member, and the third support member may be labelled with different fluorescent dyes. In some embodiments, the first labeling agent, the second labeling agent, and the third labeling agent are biotin.

In any of the methods described herein, the incubating step (b) can be performed at a temperature between about 20° C. and about 65° C. (e.g., between about 40° C. and about 65° C.). In some embodiments, the washing step (c) is performed at a temperature between about 20° C. and about 65° C. (e.g., between about 40° C. and about 65° C.). Alternatively or in addition, the washing step (c) is performed at least three times.

In some embodiments, the measuring step (d) is performed using an enzyme conjugated to streptavidin. In some embodiments, the measuring step (d) is performed by a single molecule array assay, for example Single Molecule Array (SiMoA™).

In some embodiments, the method is free of additional capture probes that are complementary to a segment of the first target nucleic acid. In some embodiments, the method is free of additional detection probes that are complementary to a segment of the first target nucleic acid.

In another aspect, the present disclosure provides a multiplex assay for detecting multiple short target nucleic acids, comprising: (i) providing a sample suspected of containing multiple target short nucleic acids, each of which is about 15-50-nucleotides in length, (ii) providing multiple sets of probes, each of which includes a capture probe immobilized on a support member and a detection probe conjugated to a labeling agent, the capture probe and the detection probe being complementary to different portions of a target short nucleic acid, wherein the multiple sets of probes are for detection of different target short nucleic acids, (iii) incubating the sample with the multiple sets of probes to form multiple complexes each containing a target short nucleic acid and a set of probes, (iv) washing the multiple complexes to remove unbound detection probes, (v) measuring signals released, directly or indirectly, from the labeling agents in the complexes; and (vi) determining presence or levels of the multiple short target nucleic acids based on the intensity of the signals detected in step (v).

In some embodiments, the multiple short target nucleic acids are about 18-25-nucleotides in length. In some instances, the multiple short target nucleic acids are RNA, e.g., microRNAs. In one example, the short target nucleic acids are mature microRNAs. In some embodiments, in each probe set, the nucleotide sequence of the capture probe that is complementary to a portion of a target short nucleic acid and the nucleotide sequence of the detection probe that is complementary to a portion of a target short nucleic differ in length by less than or equal to about 5 nucleotides. In some embodiments, the multiple target short nucleic acids are not enriched or amplified prior to step (i).

The sample suspected of containing a multiple target short nucleic acids can be a biological sample, which may be obtained from a human subject. In one example, the sample is serum (e.g., serum obtained from a human subject).

In some embodiments, the capture probe, the detection probe, or both comprise one or more locked nucleic acids (LNAs). In some embodiments, in each probe set, the capture probe and the detection probe have low cross-reactivity in the absence of the target short nucleic acid. In some instances, the capture probe and the detection probe collectively are complementary to the whole length of the target short nucleic acid. In some embodiments, in each probe set, the support member is a magnetic bead. In each probe set, the support member may be labeled by a fluorescent dye and different probe sets may contain support members labeled by different fluorescent dyes. In some embodiments, in each probe set, the labeling agent is biotin.

In some embodiments, the incubating step (iii) is performed at a temperature between about 20° C. and about 65° C. (e.g., between about 40° C. and about 65° C.). In some embodiments, the washing step (iv) is performed at a temperature between about 20° C. and about 65° C. (e.g., between about 40° C. and about 65° C.). In some instances, the washing step (iv) is performed at least three times.

In some embodiments, the measuring step (v) is performed using an enzyme conjugated to streptavidin. In some embodiments, the measuring step (v) is performed by a single molecule array assay, for example Single Molecule Array (SiMoA™).

Also within the scope of the present disclosure is a kit for detecting a target nucleic acid, such as a microRNA. The kit may comprise: (i) a capture probe immobilized on a support member; and (ii) a detection probe conjugated to a labelling agent. The target nucleic acid is about 15-50-nucleotides in length. Further, the capture probe and the detection probe each comprise a nucleotide sequence that is complementary to a first segment of the target nucleic acid and a second segment of the target nucleic acid, respectively. The first segment and second segment of the nucleic acid do not overlap.

In some examples, the support member to which the capture probe is attached is a magnetic bead. Alternatively or in addition, the labelling agent conjugated to the detection probe is biotin. In some examples, the capture probe, the detection probe, or both have a melting temperature ranging from about 30° C. and about 90° C. In some embodiments, the melting temperature of the capture probe differs from that of the detection probe by up to about 40° C. In some examples, the target nucleic acid is about 18-25-nucleotides in length.

In some embodiments, the nucleotide sequence of the capture probe that is complementary to the first segment is about 8-30-nucleotides in length and/or the nucleotide sequence of the detection probe that is complementary to the second segment is about 8-30-nucleotides in length. The nucleotide sequence of the capture probe that is complementary to the first segment and the nucleotide sequence of the detection probe that is complementary to the second segment may differ in length by less than or equal to about 5 nucleotides. In some examples, the capture probe and the detection probe collectively are complementary to the whole length of the target nucleic acid. In some embodiments, the capture probe and/or the detection probe comprises one or more locked nucleic acids (LNAs).

In another aspect, the present disclosure provides a kit for detecting multiple short target nucleic acids, comprising multiple sets of probes, each of which comprises: (i) a capture probe immobilized on a support member; and (ii) a detection probe conjugated to a labelling agent;

wherein each of the short target nucleic acids is about 15-50-nucleotides in length. Further, in each probe set, the capture probe and the detection probe are complementary to different portions of a short target nucleic acid. The multiple sets of probes are for detection of different target nucleic acids.

In some embodiments, in each probe set, the support member is a magnetic bead. In some examples, in each probe set, the labelling agent is biotin.

In some embodiments, in each probe set, the capture probe and the detection probe collectively are complementary to the whole length of the short target nucleic acid. In each probe set, the capture probe and/or the detection probe may comprise one or more locked nucleic acids (LNAs). In some embodiments, in each probe set, the nucleotide sequence of the capture probe that is complementary to the short target nucleic acid and the nucleotide sequence of the detection probe that is complementary to the short target nucleic acid differ in length by less than or equal to about 5 nucleotides. In each probe set, the capture probe, the detection probe, or both may have a melting temperature ranging from about 30° C. and about 90° C. In some examples, in at least one probe set, the melting temperature of the capture probe differs from that of the detection probe by up to about 40° C.

In some embodiments, in each probe set, the nucleotide sequence of the capture probe that is complementary to a short target nucleic acid is about 8-30-nucleotides in length and/or the nucleotide sequence of the detection probe that is complementary to the short target nucleic acid is about 8-30-nucleotides in length. In some examples, each of the short target nucleic acids is about 18-25-nucleotides in length.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a schematic diagram of an exemplary sandwich protocol for detecting nucleic acids. In this example, capture probes are covalently coupled to beads and incubated with target microRNA and biotinylated detection probes to form a sandwich complex. After washing, the beads are incubated with streptavidin-β-D-galactosidase (SBG) enzyme and resorufin-β-galactopyranoside (RGP) substrate, which produces a fluorescent product.

FIG. 2 is a schematic of an exemplary single molecule array assay. In this example, as shown in the side view, after incubation of the beads with the enzyme and the fluorogenic substrate, the beads suspended in fluorogenic substrate are loaded onto an array of femtoliter-size wells. After loading, the wells are sealed with oil, resulting in an array of isolated reaction chambers each of which contained either zero or one bead. If the enzyme was present on a bead, it generates a fluorescent product resulting in a detectable fluorescent signal. The array is imaged and analyzed to determine the total number of beads, and the number of wells with a detectable signal (“active wells”) is counted to calculate the average enzyme per bead (AEB). As shown in the top view, the number of active wells increased with increasing target concentration.

FIGS. 3A-3F are calibration curves for exemplary miRNAs. FIG. 3A is a calibration curve for miR-16, which had a limit of detection (LOD) of 0.76 fM. FIG. 3B is a calibration curve for miR-21, which had a LOD of 1.6 fM. FIG. 3C is a calibration curve for miR-141, which had a LOD of 0.58 fM. FIG. 3D is a calibration curve for miR-25, which had a LOD of 27.34 fM. FIG. 3E is a calibration curve for miR-126, which had a LOD of 8.94 fM. FIG. 3F is a calibration curve for miR-155, which had a LOD of 4.37 fM. LODs were calculated as three standard deviations above the blank for each assay.

FIGS. 4A-4B are (4A) a graph showing the results of a multiplex assay for the direct detection of three different miRNAs (i.e., miR-21, miR-141 and miR-16) and (4B) and the cross-reactivity of the multiplex assay in the presence of only miR-21, only miR-141, and only miR-16. The multiplex assay used an exemplary sandwich protocol and a single molecule array assay as described herein.

FIGS. 5A-5C are (5A) a chart showing the target nucleic acid, capture, and detection probe sequences used in a multiplex assay for the detection of let-7a, let-7b, and let-7c, (5B) a graph showing the results of a multiplex assay, and (5C) a graph and table showing the cross-reactivity of the multiplex assay in the presence of only let-7a, only let-7b, and only let-7c. The multiplex assay was performed using an exemplary sandwich protocol and a single molecule array assay as described herein. Spike-in concentrations for each of let-7a, let-7b, and let-7c in the samples (S1-S12) are given in the table. In FIG. 5A, sequences correspond to SEQ ID NOs: 64-70 from top to bottom.

FIGS. 6A-6B are (6A) a graph showing the direct detection of miR-21 spiked into human serum at varying dilutions and (6B) a graph showing the direct detection of miR-141 spiked into human serum at varying dilutions.

FIGS. 7A-7B are (7A) a graph of AEB versus total RNA concentration for the direct detection of miR-21, miR-141 and miR-16 in a total RNA sample in a multiplex assay and (7B) a graph showing the comparison between miR-21, miR-141 and miR-16 detected using an exemplary assay described herein and RT-qPCR.

FIG. 8 is a graph depicting the cross-reactivity of various let-7c capture probes. Each probe was tested against 0 fM, 1 fM, 10 fM, and 100 fM of let-7c, as well as 100 fM of let-7b. Measurements were obtained in duplicate. Incubation was four hours at room temperature.

FIG. 9 is a graph of AEB for let-7c probes 12, 17, 18, and 19. Each probe was tested against 0 fM, 1 fM, 10 fM, and 100 fM of let-7c, as well as 100 fM of let-7b. Panel A shows incubation performed at 60° C. for two hours. Panel B shows incubation performed at 65° C. for two hours. Panel C shows incubation performed for one hour at 60° C. Measurements were obtained in duplicate.

FIG. 10 is a graph of the raw multiplex data prior to correction of signal due to cross-reactivity. Each target miRNA was spiked individually to determine cross-reactivity for each plex.

FIG. 11 is a graph of the distribution of the number of mismatches in pairwise alignments between probes used in Example 1 and the broader human miRNA population. In total, 16 probes compared against 2,588 miRNA sequences gave 41,408 pairwise alignments. Panel A shows the resulting distribution and that the probes in Example 1 have low complementarity with off-target miRNA biomarkers. Panel B shows a heatmap showing the frequency of mismatches for each of the probes used in Example 1.

FIG. 12 is a graph and chart of the frequency and number of mismatches for each human miRNA in miRbase (from the “mature.fa” listing).

FIG. 13 is a graph of the distribution of calculated melting temperatures for putative probes derived from human mature miRNA. The white bars represent probes derived from miRbase and the grey bars represent probes used in Example 1.

### DETAILED DESCRIPTION OF THE INVENTION

Nucleic acids, such as microRNAs, are a promising class of biomarkers due to their association with various types of diseases, including cancer. However, current methods for nucleic acid detection, such as microRNA detection, often require lengthy sample preparation and/or pre-amplification steps, which would bias the results.

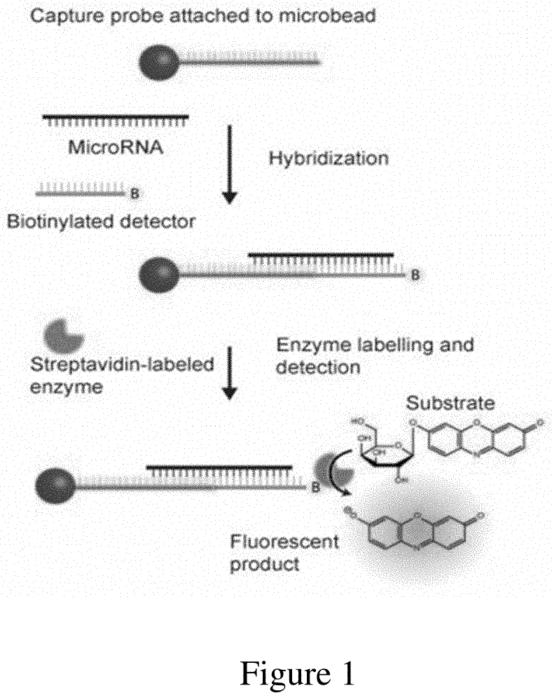

Described herein are ultrasensitive assay methods for detecting short nucleic acids such as microRNAs (e.g., in mature form), kits for performing such assay methods, and application of the assay methods in both diagnostic and non-diagnostic settings. The ultrasensitive assays aim at solving problems associated with conventional detecting assays for detecting microRNAs, such as those noted above. The assay methods described herein can be used for direct detection of short nucleic acids, such as microRNAs, at subfemtomolar concentration levels. These ultrasensitive assays may be label free, simple, and/or do not require time-consuming pre-amplification steps. Unexpectedly, an exemplary assay, involving the sandwich protocol described herein and shown FIG. 1, was applied to successfully detect single molecules of microRNAs with high sensitivity (limits of detection [LODs] ranging from below 1 femtomolar to 30 femtomolar) and specificity (distinguishing microRNAs with a single nucleotide mismatch). This assay was also successfully used to detect various microRNAs in several exemplary matrices, including human serum and total RNA samples derived from cell lysates. Further, it has been demonstrated that an exemplary sandwich protocol described herein can be used to detect multiple target microRNAs at substantially the same time with high sensitivity and specificity. The high sensitivity, simple workflow, and multiplex capability of this technique represent excellent advantages for nucleic acid-based (e.g., microRNA-based) diagnostics of human diseases. The present assay can also be used for other purposes, such as for research purposes.

### I. Ultrasensitive Assay Methods for Detecting Nucleic Acids

Described herein are methods to detect the presence and/or measure the levels of short nucleic acids, such as mature microRNAs, in a sample. A short nucleic acid as described herein refers to a nucleic acid molecule (DNA or RNA) having up to 80 nucleotides in length. In some examples, a short nucleic acid to be measured in a method described herein may contain 15-80 nucleotides in length (e.g., 15-60 nts, 15-50 nts, 18-30 nts, or 18-25 nts). In some embodiments, the ultrasensitive assay may adopt a sandwich protocol as illustrated in FIG. 1. Such an assay can be performed in a sandwich format involving the use of a capture probe and a detection probe. The capture probe and a detection probe may be hybridized with the target nucleic acid in the sample in a single assay step. In such cases, hybridization of the capture probe and the detection probe may occur at substantially the same temperature. In some cases, two different hybridization temperatures are not necessary to promote hybridization of the capture probe and the detection probe to the target nucleic acid in a sample. The capture probe can be immobilized on a support member and the detection probe can be conjugated to a labeling agent, which may release, directly or indirectly, a signal. Detection of the signal or measuring the intensity of the signal can be relied on to determine the presence and/or level of the target nucleic acid. In some examples, a single molecule array assay such as SiMoA™ technique may be used for detection. SiMoA™ is based on a conventional enzyme assay but is capable of detecting single biomolecules. The methods of the present disclosure may be employed for the detection and/or quantification of nucleic acids in a sample.

(a) Capture Probe and Detection Probe

The capture probe and detection probe for use in the ultrasensitive assay methods described herein are oligonucleotides (single-strand DNA or RNA molecules) that are complementary (partially or completely) to a region of a target short nucleic acid. In some examples, the region of a target nucleic acid that is complementary to the capture probe does not overlap with the region of the target nucleic acid that is complementary to the detection probe. In some examples, the capture probe and the detection probe, taken together, are complementary to the whole target nucleic acid. See, e.g., FIG. 1. For example, the capture probe may be complementary to the 5′ end portion of the target nucleic acid and the detection probe may be complementary to the remaining 3′ end portion of the target nucleic, or vice versa. In other examples, the capture and detection probe, taken together, are complementary to a portion of the target nucleic acid.

“Complementary,” as used herein, refers to the nucleobase complementarity commonly known in the art. For example, adenine is complementary to thymine (in DNA) or uracil in

RNA; and guanine is complementary to cytosine. “Sequence complementarity”, or “nucleic acid sequences being complementary to one another”, as used herein, means when the two nucleic acid molecules are aligned antiparallel to each other, the nucleotide bases at each position, or at most positions in the sequences are complementary, and that the two nucleic acid molecules can hybridize and form a complex under suitable conditions, e.g., hybridization temperature. As known in the art, a sequence complementarity needs not be 100% for the two nucleic acid molecules to hybridize and form a complex. The sequence complementarity between the capture probe (or the detection probe described herein) and the target nucleic acid may be at least 80% complementary to the corresponding region in the target nucleic acid. In some embodiments, the capture probe contains a fragment that is at least 80% (e.g., 85%, 90%, 95%, 98%, or 100%) to the first segment of the target nucleic acid.

Either the capture probe or the detection probe, or both may contain up to 100 nucleotides (e.g., up to 80 nt, 60 nt, 50 nt, 30 nt, or 20 nt). In some embodiments, the capture probe, the detection probe, or both may be 8-50 nucleotides in length, e.g., 8-40, 8-30, 10-30, 8-20, or 10-20 nucleotides in length. In some examples, the capture probe, the detection probe, or both may be 8, 9, 10, 11, 12, 13, 14, 15, 16, 17, 18, 19, 20, 21, 22, 23, 24, 25, 26, 27, 28, 29, or 30 nucleotides in length. In some examples, the whole molecule of the capture probe or the detection probe is complementary to a portion of a target nucleic acid. In other examples, a fragment of the capture probe or the detection probe is complementary to a portion of a target nucleic acid. For example, a capture probe may contain a linker (e.g., a poly A or poly T linker) for attaching to a support member (see details below). Alternatively or in addition, a detection probe may contain such a linker for conjugating with a labelling agent (see details below). The fragment of a capture probe or a detection probe that is complementary to a portion of a target nucleic acid may be located at the 5′ end of the probe, the 3′ end of the probe, or in the middle of the probe. In some embodiments, the fragment of the capture probe that is complementary to the target nucleic acid may be at least 8-nucleotide long (e.g., at least 10, at least 12, 15, 18, 20, or 25-nucleotide long). Alternatively or in addition, the fragment of the detection probe that is complementary to the target nucleic acid may be at least 8-nucleotide long (e.g., at least 10, at least 12, 15, 18, 20, or 25-nucleotide long). In some embodiments, the length of the fragment in a capture probe that is complementary to a portion of a target nucleic acid and that of the fragment in a detection probe that is complementary to a portion of the target nucleic acid are substantially similar, e.g., the difference is less than 5 nucleotides (for example, less than 4 nucleotides, less than 3 nucleotides, less than 2 nucleotides, less than 1 nucleotides, or identical).

In some embodiments, the capture probe, the detection probe, or both comprise one or more modified nucleotides, for example, containing nucleotides modified by a 2′-O-methoxyl group, a 2′-O-methoxyethyl group, and/or a phosphorothioate group. In some examples the capture probe, the detection probe, or both comprise one or more locked nucleic acids (LNAs). An LNA, often referred to as inaccessible RNA, is a modified RNA nucleotide, in which the ribose moiety is modified with an extra bridge connecting the 2′ oxygen and 4′ carbon. This bridge “locks” the ribose in the 3′-endo (North) conformation, which is often found in the A-form complexes. LNA nucleotides can be used in both DNA and RNA probes. In some examples, up to 50% (e.g., 40%, 30%, 20%, or 10%) of the nucleotides in the probe are LNAs. In some examples, a capture probe or a detection probe may comprise 10, 8, 6, 5, 4, 3, 2, or 1 LNA. Introducing LNAs into the capture and/or detection probe can enhance the melting temperatures of the probes such that the hybridization step (discussed in detail below) may be performed at an elevated temperature, which would improve specificity of the assay methods described herein.

Either the capture probe or the detection probe, or both may have a melting temperature of up to 90° C. (e.g., up to 85° C., 80° C., 75° C., 70° C., 65° C., 60° C., 55° C., 50° C., or 45° C.). In some embodiments, the melting temperature of the capture probe, the detection probe, or both may be between about 10° C. and about 70° C., e.g., between about 10° C. and about 70° C., between about 10° C. and about 60° C., between about 15° C. and about 60° C., or between about 15° C. and 55° C. In some embodiments, the difference in the melting temperature between the capture probe and the detection probe may be relatively small. The relatively small difference in melting temperature may contribute, at least in part, to hybridization of the capture probe and detection probe at substantially the same temperature. In some embodiments, the difference in melting temperature may be up to 40° C. (e.g., up to 35° C., 30° C., 25° C., or 20° C.). For instance, the difference in melting temperature between the capture probe and the detection probe may be between about 0° C. and 40° C., between about 0° C. and 35° C., between about 0° C. and 30° C., or between about 0° C. and 20° C. In general, the term “about” means within an acceptable error range for the particular value as determined by one of ordinary skill in the art, which will depend in part on how the value is measured or determined, i.e., the limitations of the measurement system. For example, in regard to temperature, “about” can mean within an acceptable standard deviation, per the practice in the art. “About” can mean a range of up to ±20%, preferably up to ±10%, more preferably up to ±5%, and more preferably still up to ±1% of a given value.

The capture probe and the detection probe may have relatively low cross-reactivity in the absence of the target nucleic acid. In some embodiments, the cross-reactivity is less than or equal to about 20% (e.g., less than or equal to about 18%, 15%, 12%, 10%, 8%, 5%, or 2%). In some instances, the cross-reactivity is less than or equal to about 10%. Percent cross-reactivity is the percent of the signal above baseline in the absence of the entity that cross-reacts.

Both the capture probe and the detection probe can be designed based on the sequence of the target nucleic acid and be prepared via conventional methods, for example, chemical synthesis or in vitro transcription.

The capture probe as described herein can be immobilized on a support member via a conventional method. As used herein, “immobilized” means attached, bound, or affixed, covalently or non-covalently, so as to prevent dissociation or loss of the capture probe, but does not require absolute immobility with respect to either the capture probe or the support member. A support member can be a solid or semi-solid member with a surface that can be used to specifically attach, bind or otherwise capture a nucleotide probe (e.g., the capture probe of the present disclosure), such that the nucleotide probe becomes immobilized with respect to the support member.

The support member of the present disclosure may be fabricated from one or more suitable materials, for example, plastics or synthetic polymers (e.g., polyethylene, polypropylene, polystyrene, polyamide, polyurethane, phenolic polymers, or nitrocellulose), naturally derived polymers (e.g., latex rubber, polysaccharides, polypeptides), composite materials, ceramics, silica or silica-based materials, carbon, metals or metal compounds (e.g., comprising gold, silver, steel, aluminum, or copper), inorganic glasses, silica, and a variety of other suitable materials. Non-limiting examples of potentially suitable configurations include beads (e.g., magnetic beads), tubes (e.g., nanotubes), plates, disks, dipsticks, chips, microchips, coverslips, or the like.

The surface of the support member of the present disclosure, may comprise any molecule, other chemical/biological entity, or solid support modification disposed upon the solid support that can be used to specifically attach, bind or otherwise capture a nucleic acid molecule (e.g., a capture probe). Surface compositions that may be used to immobilize a nucleic acid molecule can be readily found in the art. For example, the surface may comprise a complementary nucleic acid or a nucleic acid binding protein. Thus, the linkage between the nucleic acid to be immobilized (e.g., the capture probe of the present disclosure) and the surface may comprise one or more chemical or physical (e.g., non-specific attachment via van der Waals forces, hydrogen bonding, electrostatic interactions, hydrophobic/hydrophilic interactions; etc.) bonds and/or chemical linkers providing such bond(s). Alternatively, the surface of the support member may comprise reactive functional groups that are capable of forming covalent bonds with the nucleic acid molecules to immobilized. In some embodiments, the functional groups are chemical functionalities. That is, the binding surface may be derivatized such that a chemical functionality is presented at the binding surface which can react with a chemical functionality on nucleic acid to be capture, resulting in attachment. Examples of functional groups for attachment that may be useful include, but are not limited to, amino groups, carboxy groups, epoxide groups, maleimide groups, oxo groups, and thiol groups. Functional groups can be attached, either directly or through the use of a linker, the combination of which is sometimes referred to herein as a “crosslinker.” Crosslinkers for attaching nucleic acid molecules to a support member are known in the art; for example, homo-or hetero-bifunctional crosslinkers as are well known (e.g., see 1994 Pierce Chemical Company catalog, technical section on crosslinkers, pages 155-200, or “Bioconjugate Techniques” by Greg T. Hermanson, Academic Press, 1996). Non-limiting example of crosslinkers include alkyl groups (including substituted alkyl groups and alkyl groups containing heteroatom moieties), esters, amide, amine, epoxy groups and ethylene glycol and derivatives. A linker may also be a sulfone group, forming a sulfonamide. In some embodiments, the functional group is a light-activated functional group. That is, the functional group can be activated by light to attach the capture component to the capture object surface. One example is PhotoLinkTM technology available from SurModics, Inc. in Eden Prairie, Minn.

It is to be understood that the examples provided herein on the support member and the surface composition are not meant to be limiting. Any support members that are known in the art to be suitable for immobilization of nucleic acid molecules may be used in accordance with the present disclosure.

The detection probe may be conjugated with a labeling agent. “Conjugated”, as used herein, means the labeling agent is attached to the detection probe, covalently or non-covalently. The labeling agent can be any molecule, particle, or the like, that facilitates detection, directly or indirectly, using a suitable detection technique. In the case of direct detection, the labeling agent may be a molecule or moiety capable of releasing a signal that can be directly interrogated and/or detected (e.g., a fluorescent label or a dye). In a first non-limiting case of indirect detection, the labeling agent may be a molecule or moiety capable of converting a substrate (e.g., an enzyme) to a product that is capable of releasing a detectable signal. For example, the labeling agent may be a luciferase, which converts luciferin to oxyluciferin to emit detectable lights. In another non-limiting case of indirect detection, the labeling agent is a binding ligand to a molecule or moiety capable of converting a substrate (e.g., an enzyme), wherein the converted substrate releases detectable signals. For example, as illustrated in FIG. 1, the labeling agent is a biotin, which is a binding ligand to a streptavidin-β-D-galactosidase (SBG) fusion protein. The SBG enzyme is able to convert its substrate resorufin-β-galactopyranoside (RGP) to a product that has a detectable fluorescent signal.

In some embodiments, a fluorescent label is used as the labeling agent. Examples include, but are not limited to, fluorescein, isothiocyanate, rhodamine, phycoerythrin, phycocyanin, allophycocyanin, o-phthaldehyde, fluorescamine and fluorescent metals such as ¹⁵²Eu or other metals from the lanthanide series, CYE dyes, and fluorescent proteins such as eGFP, eYFP, eCFP, mKate2, mCherry, mPlum, mGrape2, mRaspberry, mGrape1, mStrawberry, mTangerine, mBanana, and mHoneydrew.

Other exemplary labelling agents include, but are not limited to, biotin, phosphorescent labels, chemiluminescent labels or bioluminescent labels (such as luminal, isoluminol, theromatic acridinium ester, imidazole, acridinium salts, oxalate ester, and dioxetane), radio-isotopes (such as ³H, ¹²⁵I, ³²P, ³⁵S, ¹⁴C, ⁵¹Cr, ³⁶Cl, ⁵⁷Co, ⁵⁸Co, ⁵⁹Fe, and ⁷⁵Se), metals, metal chelates or metallic cations (for example metallic cations such as ⁹⁹mTc, ¹²³I, ¹¹¹In, ¹³¹I, ⁹⁷Ru, ⁶⁷Cu, ⁶⁷Ga, and ₆₈Ga. Other examples include chromophores and enzymes (e.g., malate dehydrogenase, staphylococcal nuclease, delta-V-steroid isomerase, yeast alcohol dehydrogenase, alpha-glycerophosphate dehydrogenase, triose phosphate isomerase, peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase).

(b) Hybridization

The ultrasensitive assays described herein may involve a hybridization step, in which the capture probe and the detection probe form complexes with a target nucleic acid of interest. As described herein, the capture probe and detection probe may be designed such that suitable hybridization of both the capture probe and the detection probe occur at substantially the same temperature. In such cases, hybridization of the capture probe and the detection probe may occur in a single step. In some examples, the method may comprise a single hybridization step. In the hybridization step, a target nucleic acid is hybridized to a capture probe and a detection probe as described herein under at a hybridization temperature to form a complex. See, e.g., FIG. 1.

Hybridization refers to the ability of complementary single-stranded DNA or RNA to form a complex. The hybridization step of the ultrasensitive assay described herein can be performed under suitable hybridization conditions, which are within the knowledge of those skilled in the art. Hybridization conditions resulting in particular degrees of stringency will vary depending upon the nature of the hybridization method and the composition and length of the hybridizing nucleic acid sequences. Generally, the temperature of hybridization and the ionic strength of the hybridization buffer will determine the stringency of hybridization. Calculations regarding hybridization conditions for attaining particular degrees of stringency are well known in the art, for example, described in Sambrook et al., (1989) Molecular Cloning, second edition, Cold Spring Harbor Laboratory, Plainview, N.Y. (chapters 9 and 11). The hybridization temperature of the assay methods described herein can be determined based on various factors, for example, the length of the complementary regions between the capture/detection probe and the target nucleic acid, the composition of the complementary regions (e.g., G/C content), and the stringency needed, which are within the knowledge of those skilled in the art.

The hybridization step may be performed under a suitable temperature, e.g., a temperature under which the capture probe and the detection probe form complexes with a target nucleic acid of interest with high specificity and form little or no complexes with other short nucleic acids, even those that share sequence homology with the target nucleic acid of interest. Such a suitable hybridization temperature can be determined based on various factors as known to those skilled in the art, for example, melting temperatures of the capture and detection probes, length of the target nucleic acid, and presence of homologous non-target nucleic acids in the same sample. In some embodiments, the hybridization temperature may be less than the melting temperature of the capture probe, detection probe, or both. In some embodiments, hybridization may be carried out at a temperature between about 40° C. and about 65° C., for example, about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In one example, the hybridization temperature ranges from about 40° C. to about 65° C. or from about 50° C. to about 65° C.

Other hybridization conditions, such as ion strength, can be determined based on the factors described above. In the hybridization step, the capture probe, detection probe, and the target nucleic acid form a double-stranded nucleic acid complex, in which the fragment of the capture probe that is complementary to the target nucleic acid forms base pairs with the corresponding segment in the target nucleic acid and the fragment of the detection probe that is complementary to the target nucleic acid forms base pairs with the corresponding segment in the target nucleic acid. See, e.g., FIG. 1. In some embodiments, the segment of the target nucleic acid that is complementary to the capture probe and the segment of the target nucleic acid that is complementary to the detection probe have similar lengths. In some examples, the difference in length between the segment of the target nucleic acid that is complementary to capture probe and the segment that is complementary to the detection probe may be less than or equal to 10 nucleotides (e.g., up to 8 nt, 6 nt, 5 nt, 3 nt, 2 nt, or 1 nt). In one example, the difference may be less than or equal to 5 nucleotides.

(c) Washing

The ultrasensitive assays described herein may involve a washing step. For instance, after the hybridization step, the reaction mixture can be washed any suitable number of times to remove unbound components (e.g., capture probe and/or detection probe). In some examples, the washing step may be performed at least two times, at least three times, at least four times, at least five times, at least six times, at least seven times, or at least eight times. In one example, the washing step is performed at least three times (e.g., three to eight times). In another example, a single wash step is performed.

In some embodiments, one or more wash steps may be performed at an elevated temperature. For instance, the wash step may be carried out at a temperature between about 20° C. and about 65° C., for example, about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In one example, the wash temperature ranges from about 40° C. to about 65° C. or from about 45° C. to about 55° C.

(d) Detection

The target nucleic acid-containing complex can then be detected via a suitable method, which depends on the type of labeling agent conjugated to the detection probe in the complex. Any detection methods known in the art and are suitable for the labeling agent of choice may be used. In some embodiments, the detection method may use an enzyme (e.g., enzyme conjugated to streptavidin). In some embodiments, the detection method involves a single molecule array assay (for example, the SiMoATM technology) known in the art. Exemplary single molecule array assays have been described previously, for example, U.S. Pat. Nos. 8,460,879, 8,460,878, 8,492,098, 8,222,047, 8,236,574, 8,415,171, US2010-0075862, US2010-0075439, US2010-0075355, US 2011-0212462, US 2012-0196774, US 2011-0245097, WO 2009/029073, WO2010/039179, WO2011/109364, WO2011/109372, WO2011/109379, WO/2014/113502, the relevant disclosures of each of which are incorporated by reference herein for purposes or subject matter referenced herein.

(e) Multiplex Assays

In some embodiments, the ultrasensitive assay method is used to detect the presence or measure the level of two or more (e.g., three or more, four or more, or five or more) target nucleic acids in a sample. In some cases, the ultrasensitive assay method may be a multiplex assay in which the presence or measure of the level of more than one target nucleic acid is measured in a single performance of the assay. In such cases, at least some (e.g., all) of the target nucleic acids are measured at one time. It has been surprisingly found that the methods, described herein with respect to the detection of a single target nucleic acid (single-plex methods), can be used to achieve specific and sensitive (e.g., average LODs of less than 15 femtomolar) detection and quantification of target nucleic acids in a multiplex assay.

In some embodiments, the ultrasensitive multiplex assay may utilize the sandwich protocol, illustrated in FIG. 1, for each target nucleic in the sample. In such cases, a multiplex assay for detecting multiple target nucleic acids may utilize a different capture probe and detection probe set for each target nucleic acid. As described herein with respect to the single-plex assay, the capture probe and the detection probe for a given target nucleic acid in a multiplex assay may also be complementary to different portions of the target nucleic acid. In some embodiments, the capture probe and the detection probe for a given target nucleic acid are not complementary to another and/or all other nucleic acids (e.g., non-target nucleic acid) in the sample. In general, each set of capture and detection probes has a relatively low cross-reactivity with another set (e.g., all other sets) of capture and detection probes. Each set of capture and detection probes may have a relatively low cross-reactivity in the absence of the target nucleic acid.

Each capture probe in the multiplex assay can be immobilized on a support member and/or each detection probe can be conjugated to a labeling agent, as described herein. In some embodiments, the capture probe and/or detection probe for each target nucleic acid may be differently labeled, such that a unique signal can be associated with each target nucleic acid. In some instances, the support members may differ for at least some (e.g., each) set of probes. For example, each set of probes comprises a support member labelled with a distinct label (e.g., dye such as a fluorescent dye). In such cases, each support member in the multiplex assay comprises a different label (e.g., fluorescent dye). In some embodiments, the labeling agent on each detection probe in the multiplex assay differs.

In some embodiments, a multiplex assay may comprise incubating the sample with the multiple sets of probes. For example, a multiplex assay for a sample suspected of containing two or more (e.g., three, four, five, or more) target nucleic acids may comprise incubating the sample with at least a first set of probes (e.g., a first capture probe and a first detection probe) and second set of probes (e.g., a second capture probe and a second detection probe). Additional target nucleic acids may be detected by incubating the sample with additional sets of probes (e.g., a third set of probes, a fourth set of probes, a fifth set of probes, etc.). In general, any suitable number of target nucleic acids (e.g., two, three, four, five or more) may be detected in a sample using the appropriate number of probe sets. In some embodiments, the incubation of the sample with the multiple sets of probes occurs in a single step. For example, multiple sets of probes may be incubated with a single sample at substantially the same time.

In general, the sets of probes and sample may be incubated at a hybridization temperature that promotes the formation of complexes between each target nucleic acid and its associated probe set. For example, in a sample suspected of containing three target nucleic acids, a first capture probe and a first detection probe may form a first complex with a first target nucleic, a second capture probe and a second detection probe may form a second complex with a second target nucleic, and a third capture probe and a third detection probe may form a third complex with a third target nucleic. In some embodiments, the hybridization temperature for each target nucleic acid may be substantially the same. In such cases, the multiplex assay has a single hybridization step. The hybridization temperature may be as described herein with respect to detection of a single target nucleic acid.

After the hybridization step, the multiplex reaction mixture can be washed any suitable number of times to remove unbound components (e.g., multiple capture probe, multiple detection probe), as described herein. The target nucleic acid-containing complexes may then be detected via any suitable method that allows the signal associated with each target nucleic acid to be distinguished. In some embodiments, the capture probe and/or detection probe for each target nucleic acid may be differently labeled, such that a unique signal may be associated with each target nucleic acid. For instance, the support member for each capture probe may be labeled with a different fluorescent dye. In some such cases, a target nucleic acid-containing complex may be distinguished from other target nucleic acid-containing complexes based at least in part on the fluorescent dye. In some instances, the labeling agent for each detection probe may be different. In some such cases, a target nucleic acid-containing complex may be distinguished from other target nucleic acid-containing complexes based at least in part on the different labeling agents. In some instances, both the support member and the labeling agent may differ between set of probes.

In some embodiments, detection in a multiplex assay comprises measuring signals released, directly or indirectly, from the labeling agents in the complexes and determining the presence or levels of the multiple target nucleic acids based on the intensity of the detected signals. For example, in a sample suspected of containing three target nucleic acids, detection may comprise (i) measuring a first signal released, directly or indirectly, from the first labeling agent in the first complex, and determining the presence or a level of the first target nucleic acid in the sample based on the intensity of the first signal; (ii) measuring a second signal released, directly or indirectly, from the second labeling agent in the second complex, and determining the presence or a level of the second target nucleic acid in the sample based on the intensity of the second signal; and (iii) measuring a third signal released, directly or indirectly, from the third labeling agent in the third complex, and determining the presence or a level of the third target nucleic acid in the sample based on the intensity of the third signal.

In some embodiments, the ultrasensitive multiplex assay methods may allow for the specific and sensitive detection and quantification of highly homologous target nucleic acids. For example, the target nucleic acids may be at least 80% (e.g., at least 90%, at least 95%, or at least 98%) identical. In some examples, the one or more homologous target nucleic acids may differ by only up to 5 nucleotides (e.g., 4, 3, 2, or 1).

(f) Applications

The ultrasensitive assay methods described herein can be used to detect the presence and/or measure the level of a nucleic acid of interest (a target nucleic acid) in a suitable sample. In some examples, the sample may be a biological sample obtained from a subject and the results obtained from the assay methods described herein may be used for diagnostic and/or prognostic purposes. In other examples, the assay methods described herein can be used in research settings for detecting presence or measuring the level of a target nucleic acid in a sample.

To measure the level (concentration) of a target nucleic acid in a sample, a calibration curve may be developed using samples containing known concentrations of the target nucleic acid molecule. The concentration of the target nucleic acid in a sample may be determined by comparison of a measured parameter to a calibration standard. In some cases, a calibration curve may be prepared, wherein the total measured signal is determined for a plurality of samples comprising the target nucleic acid at a known concentration using a substantially similar assay format. For example, the total intensity of the array, may be compared to a calibration curve to determine a measure of the concentration of the target nucleic acid in the sample. The calibration curve may be produced by completing the assay with a plurality of standardized samples of known concentration under similar conditions used to analyze test samples with unknown concentrations. A calibration curve may relate the detected signal of the target nucleic acid (and/or detection probe) with a known concentration of the target nucleic acid. The assay may then be completed on a sample containing the target nucleic acid or fragment in an unknown concentration, and signals detected from the target nucleic acid (and/or detection probe) may be compared to the calibration curve, (or a mathematical equation fitting same) to determine a measure of the concentration of the target nucleic acid in the sample.

The ultrasensitive assay methods described herein may be used to detect any nucleic acid molecule, including both DNA molecules and RNA molecules. When the target nucleic acid is a DNA molecule, a denaturing step may be performed to produce single-stranded DNA molecules. In some embodiments, the assay methods are applied to detecting short nucleic acids, for example, nucleic acids having less than 80 nucleotides (nts), e.g., less than 60 nts, less than 50 nts, less than 40 nts, less than 30 nts, less than 25 nts, or less than 20 nts. In one example, the assay methods are applied to detecting short nucleic acids having a length of about 15-50 nt (e.g., 18-25 nucleotides in length). In a particular example, the assay methods are applied for detecting microRNAs (e.g., mature microRNA). Given the high sensitivity of the ultrasensitive assay methods described herein, a target nucleic acid may not need to be pre-amplified.

In some embodiments, the ultrasensitive assay methods are applied to detect a target nucleic acid in a biological sample, which may be any sample from a biological source. Exemplary biological samples include tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments, the biological sample can be a body fluid, which can be fluid isolated from the body of an individual. For example, “body fluid” may include blood, plasma, serum, bile, saliva, urine, tears, perspiration, and the like.

The biological sample may be obtained from a subject in need of the analysis. A “subject” may be a human (i.e., male or female of any age group, for example, pediatric subject (e.g., infant, child, or adolescent) or adult subject (e.g., young adult, middle-aged adult, or senior adult). Alternatively, the subject may be a non-human animal. In certain embodiments, the non-human animal is a mammal (e.g., primate, for example, cynomolgus monkey or rhesus monkey), commercially relevant mammal (e.g., cattle, pig, horse, sheep, goat, cat, or dog), or bird (e.g., commercially relevant bird, such as chicken, duck, goose, or turkey). In other examples, the non-human animal is a fish, reptile, or amphibian. The non-human animal may be a male or female at any stage of development. The non-human animal may be a transgenic animal or genetically engineered animal. In some examples, the subject may also be a plant.

In some embodiments, the sample for analysis may contain one or more nucleic acids that are highly homologous to the target nucleic acid, e.g., at least 80%, 90%, 95%, or 98% identical to the target nucleic acid. The “percent identity” of two nucleic acids can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. In some examples, the one or more homologous nucleic acids may differ from the target nucleic acid by only up to 5 nucleotides (e.g., 4, 3, 2, or 1).

The ultrasensitive assay methods may be applied in a diagnostic/prognostic setting to detect the presence or measure the level of a nucleic acid biomarker that is associated with a target disease. For example, the methods may be used to detect/measure a specific microRNA, which may be associated with a specific disease, e.g., cancer. The methods can be used in detecting such a nucleic acid biomarker in subjects that are absent of any symptom of the disease for early stage diagnosis. The assay methods can also be used to detect nucleic acids of microorganisms for determining whether a subject has been infected by such microorganisms, for example, viruses (e.g., HBV, HCV, HPV, and HIV).

Those skilled in the art would have known that the application of the ultrasensitive assay methods described herein are not limited to diagnosis/prognosis purposes; such methods can be used to detect nucleic acids of interest for any purposes, for example, for research purposes. In some examples, the assay methods can be applied to detect a nucleic acid such as a microRNA in studies of its biological functions or in studies of bio-pathways, in which the nucleic acid is involved. Alternatively, the assay methods described herein can also be used in development of nucleic acid-based therapeutic agents.

### II. Kits for Performing the Ultrasensitive Assay Methods

The present disclosure also provides kits for use in performing the ultrasensitive assay methods described herein. Such kits may be designed for diagnostic uses or for other purposes, for example, research uses.

The kit described herein may include one or more containers housing components for performing the assay methods described herein and optionally instructions of uses. Specifically, such a kit may include one or more agents described herein (for example, a capture probe and a detection probe as described herein), along with instructions describing the intended application and the proper use of these agents. In certain embodiments, the kit may be suitable for a diagnostic purpose. For example, the kit may contain apparatus for sample collection from a patient, and/or reagents for detecting diseases associated nucleic acid molecules. Kits for research purposes may contain the components in appropriate concentrations or quantities for running various experiments.

The kit described herein may contain one or more sets of probes (e.g., 2 sets, 3 set, 4 sets, or 5 sets), each comprising a capture probe, which may be immobilized in a support member as described herein, and a detection probe, which may be conjugated with a labeling agent as also described herein. Alternatively, the kit may contain the capture probe in free form, the support member, and reagents necessary for linking the capture probe onto the surface of the support member. For example, the support member in the kit may comprise chemical reactive moieties for the covalently linking of the capture probes. Alternatively or in addition, the kit may comprise the detection probe in free form, the labeling agent, and reagents necessary for use to conjugate the labeling agent to the detection probe.

Any of the kit described herein may further comprise components needed for performing the assay methods. For example, it may contain components for use in detecting a signal released from the labeling agent, directly or indirectly. In some examples, the detection step of the assay methods involves enzyme reaction, the kit may further contain the enzyme and a suitable substrate.

Each components of the kits, where applicable, may be provided in liquid form (e.g., in solution), or in solid form, (e.g., a dry powder). In certain cases, some of the components may be constitutable or otherwise processable (e.g., to an active form), for example, by the addition of a suitable solvent or other species (for example, water or certain organic solvents), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which can also reflects approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention. Additionally, the kits may include other components depending on the specific application, as described herein.

The kits may contain any one or more of the components described herein in one or more containers. The components may be prepared sterilely, packaged in syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Ultrasensitive Assay Methods for Detecting microRNAs

### Materials and Methods

### Locked Nucleic Acid Probe Design

Locked nucleic acid (LNA) capture and detection probes were designed to be partially complementary to their intended target miRNA. The length of each probe and the placement of bases within each LNA probe were then selected based on the following criteria: (1) consistent melting temperature for both capture and detection probe; (2) strong predicted binding between each probe and the target sequence; and (3) low cross-reactivity between the capture and detection probes (in the absence of target). For each target miRNA, proposed designs for the candidate capture and detection probes were checked using the LNA Oligo Tₘ Prediction and LNA Oligo Optimizer tools on the Exiqon website. Probe designs were manually iterated to maximize the ratio of predicted target binding/predicted capture-detector binding and to ensure the secondary structure score for capture-detector hybridization remained low (below 20).

Table 1 shows the sequences for the miRNA targets, capture probes, and detection probes. In Table 1, RNA bases are proceeded by “r” (i.e. rA, rC, rG, rU), and locked nucleic acid (LNA) bases are proceeded by “+” (i.e. +A, +C, +G, +T). Amine and biotin modifications are indicated (“/5AmMC12/” and “/3Bio/”, respectively).

TABLE 1
Oligonucleotide sequences of miRNA targets, capture probes,
and detection probes.

| | |
|---|---|
| Oligonucleotides | Sequences (5′-3′) |

Synthetic miRNA targets

| | |
|---|---|
| hsa-miR16-5p | rUrArGrCrArGrCrArCrGrUrArArArUrArUrUrGrGrCrG |
| | (SEQ ID NO: 1) |
| hsa-miR21-5p | rUrArGrCrUrUrArUrCrArGrArCrUrGrArUrGrUrUrGrA |
| | (SEQ ID NO: 2) |
| hsa-miR25-3p | rCrArUrUrGrCrArCrUrUrGrUrCrUrCrGrGrUrCrUrGrA |
| | (SEQ ID NO: 3) |
| hsa-miR126-3p | rUrCrGrUrArCrCrGrUrGrArGrUrArArUrArArUrGrCrG |
| | (SEQ ID NO: 4) |
| hsa-miR141-3p | rUrArArCrArCrUrGrUrCrUrGrGrUrArArArGrArUrGrG |
| | (SEQ ID NO: 5) |
| hsa-miR155-5p | rUrUrArArUrGrCrUrArArUrCrGrUrGrArUrArGrGrGrGrU |
| | (SEQ ID NO: 6) |
| hsa-let-7a-5p | rUrGrArGrGrUrArGrUrArGrGrUrUrGrUrArUrArGrUrU |
| | (SEQ ID NO: 7) |
| hsa-let-7b-5p | rUrGrArGrGrUrArGrUrArGrGrUrUrGrUrGrUrGrGrUrU |
| | (SEQ ID NO: 8) |
| hsa-let-7c-5p | rUrGrArGrGrUrArGrUrArGrGrUrUrGrUrArUrGrGrUrU |
| | (SEQ ID NO: 9) |

Capture probes

| | |
|---|---|
| miR16-5pCapture | /5AmMC12/TTTTTTCG+CC+AA+TA+TT+T (SEQ ID NO: 10) |
| miR21-5pCapture | /5AmMC12/TTTTTT+T+CA+A+CATCAG+T (SEQ ID NO: 11) |
| miR25-3pCapture | /5AmMC12/TTTTTTT+CAGA+CCGA+GA (SEQ ID NO: 12) |
| miR126-3pCapture | /5AmMC12/TTTTTTCG+CA+T+TA+T+TAC (SEQ ID NO: 13) |
| miR141-3pCapture | /5AmMC12/TTTTTTC+CATCT+TTA+C+C (SEQ ID NO: 14) |
| miR155-5pCapture | /5AmMC12/TTTTTTA+C+CCCTATCA+C (SEQ ID NO: 15) |
| let-7a-5p | /5AmMC12/TTTTTTA+AC+TA+TA+CA+AC (SEQ ID NO: 16) |
| let-7b-5p | /5AmMC12/TTTTTTA+AC+CA+CA+CA+AC (SEQ ID NO: 17) |
| let-7c-5p | /5AmMC12/TTTTTTA+AC+CA+TA+CA+AC (SEQ ID NO: 18) |

Detection probes

| | |
|---|---|
| miR16-5pDetector | A+CGTGCTGC+TA/3Bio/ (SEQ ID NO: 19) |
| miR21-5pDetector | C+TGAT+A+AG+C+TA/3Bio/ (SEQ ID NO: 20) |
| miR25-3pDetector | +CA+A+G+TGCA+A+TG/3Bio/ (SEQ ID NO: 21) |
| miR126-3pDetector | +TCA+CGGTA+CGA/3Bio/ (SEQ ID NO: 22) |
| miR141-3pDetector | +A+GA+CA+GTG+TTA/3Bio/ (SEQ ID NO: 23) |
| miR155-5pDetector | +G+AT+TAG+CA+T+TA+A/3Bio/ (SEQ ID NO: 24) |
| let-7Detector | C+TA+CT+AC+CT+CA/3Bio/ (SEQ ID NO: 25) |

Table 2 shows that Let-7c capture probe sequences along with the melting temperature (Tₘ, as estimated by the Exiqon LNA Oligo Tm Prediction tool) and the number of LNA residues. LNA bases are proceeded by “+” (i.e. +A, +C, +G, +T), and amine modifications are indicated as “/5AmMC12/”. The sequence of let-7 detection probe is shown in Table 1.

TABLE 2
Let-7c capture probe sequences, melting temperatures,
and number of LNAs.

| | | RNA Tm | |
|---|---|---|---|
| Let-7c probes | | (° C.) | # of LNAs |
| Probe 1 | /5AmMC12/TTTTTTAACCATACAAC (SEQ ID | 25 | 0 |
| | NO: 26) | | |
| Probe 2 | /5AmMC12/TTTTTT+AA+CC+AT+AC+AA+C | 68 | 6 |
| | (SEQ ID NO: 27) | | |
| Probe 3 | /5AmMC12/TTTTTTAA+C+CATA+CAA+C | 58 | 6 |
| | (SEQ ID NO: 28) | | |
| Probe 4 | /5AmMC12/TTTTTTAACC+A+T+ACAAC (SEQ | 50 | 4 |
| | ID NO: 29) | | |
| Probe 5 | /5AmMC12/TTTTTT+A+A+CCA+TACA+A+C | 74 | 3 |
| | (SEQ ID NO: 30) | | |
| Probe 6 | /5AmMC12/TTTTTT+A+ACCA+TA+CAA+C | 60 | 6 |
| | (SEQ ID NO: 31) | | |
| Probe 7 | /5AmMC12/TTTTTTAA+CCA+TAC+AAC (SEQ | 64 | 5 |
| | ID NO: 32) | | |
| Probe 8 | /5AmMC12/TTTTTTAA+CCA+TAC+AA+C | 64 | 3 |
| | (SEQ ID NO: 33) | | |
| Probe 9 | /5AmMC12/TTTTTTAA+C+CA+TA+CAA+C | 66 | 4 |
| | (SEQ ID NO: 34) | | |
| Probe 10 | /5AmMC12/TTTTTT+A+AC+C+A+TA+C+AA+C | 64 | 5 |
| | (SEQ ID NO: 35) | | |
| Probe 11 | /5AmMC12/TTTTTT+AA+C+CA+T+A+CAA+C | 72 | 8 |
| | (SEQ ID NO: 36) | | |
| Probe 12 | /5AmMC12/TTTTTTA+AC+CA+TA+CA+AC | 68 | 7 |
| | (SEQ ID NO: 37) | | |
| Probe 13 | /5AmMC12/TTTTTTA+AC+CAT+A+CA+AC | 71 | 5 |
| | (SEQ ID NO: 38) | | |
| Probe 14 | /5AmMC12/TTTTTTA+AC+C+ATA+CA+AC | 61 | 5 |
| | (SEQ ID NO: 39) | | |
| Probe 15 | /5AmMC12/TTTTTTA+A+CC+AT+ACA+AC | 72 | 5 |
| | (SEQ ID NO: 40) | | |
| Probe 16 | /5AmMC12/TTTTTTA+A+C+C+ATA+CAAC | 66 | 5 |
| | (SEQ ID NO: 41) | | |
| Probe 17 | /5AmMC12/TTTTTTA+A+CC+AT+A+CAAC | 76 | 5 |
| | (SEQ ID NO: 42) | | |
| Probe 18 | /5AmMC12/TTTTTTA+A+C+CATA+CA+AC | 71 | 5 |
| | (SEQ ID NO: 43) | | |
| Probe 19 | /5AmMC12/TTTTTTAA+CC+AT+A+CA+AC | 72 | 5 |
| | (SEQ ID NO: 44) | | |
| Probe 20 | /5AmMC12/TTTTTTAAC+C+AT+A+CA+AC | 66 | 5 |
| | (SEQ ID NO: 45) | | |

Considerations for the complementarity and melting temperature of capture and detection probes as applied to the general population of human miRNA are described in Example 2.

### Covalent Coupling of Capture Probes to Paramagnetic Microbeads

Custom-made LNA capture probes were purchased from Exiqon. Carboxylated 2.7 μm paramagnetic beads, non-encoded for single-plex assays and dye-encoded (488 nm, 647 nm, and 700 nm) for multiplex assays, were purchased from Quanterix. 5×10⁸ beads were washed three times with 0.01 M NaOH. 1-ethyl-3-(3-dimethylaminopropyl)carbodiimide hydrochloride (EDC) and N-hydroxysulfosuccinimide (sulfo-NHS) were reconstituted in 2-(N-morpholino)ethanesulfonic acid (MES) buffer (50 mM, pH 6.0) to a final concentration of 50 mg/ml. 100 μl of each of the EDC and sulfo-NHS were added to the beads. The beads were activated on a shaker for 30 minutes. After activation, the beads were washed once with coupling buffer (1× phosphate buffered saline [PBS], 0.5 M NaCl, 0.1% Tween 20, pH 7.4). 20 nmol of the capture probe were diluted into 200 μl of coupling buffer and added to the beads. The beads were incubated at room temperature with shaking for three hours. The beads were then washed with wash buffer (1× PBS, 1% Tween 20) and incubated in 200 μl of quenching buffer (100 mM Tris-HCl, pH 7.4) with shaking for 45 minutes. The beads were washed twice with wash buffer and incubated in 200 μl of blocking buffer (1× PBS, 1% bovine serum albumin [BSA]) with shaking for 45 minutes. The beads were then washed three times with wash buffer and resuspended in a bead storage buffer (50 mM Tris-HCl, 150 mM NaCl, 10 mM ethylenediaminetetraacetic acid [EDTA], 0.1% Tween 20, and 1% BSA). The beads were counted using a Beckman-Coulter multisizer. Two three-plex Simoa assays were developed. The first three-plex assay simultaneously measured let-7a, let-7b, and let7c, which were coupled to 488 nm, 647 nm, and 700 nm dye-encoded beads, respectively. The second three-plex assay simultaneously measured miR-21, miR-141, and miR-16 coupled to 488 nm, 647 nm, and 700 nm dye-encoded beads, respectively.

Setup of miRNA Simoa Assays

Synthetic target miRNAs were purchased from IDT. Synthetic miRNAs were serially diluted in hybridization buffer (5× saline-sodium citrate [SSC] in diethyl pyrocarbonate [DEPC]-treated water) to desired concentrations. Capture beads (prepared as described above) were diluted to a concentration of 50,000 beads/μl for single-plex assays and 90,000 beads/μl for three-plex assays (with 30,000/μl beads per target) in hybridization buffer. Biotinylated LNA-modified detection probes were purchased from Exiqon and diluted to a concentration of 20 nM in hybridization buffer. 100 μl of sample, 10 μl capture beads, and 10 μl of biotinylated detection probes were added to a low binding 96 well plate (Corning, CLS3651) and incubated at 50° C., with the exception that the let-7 multiplex assay was incubated at 55° C., with shaking for two hours. The beads were then washed eight times with System Wash Buffer 1 (Quanterix), warmed to 50° C., using a microplate washer (BioTek). Streptavidin-β-galactosidase (SβG) Concentrate (Quanterix) was diluted to 200 pM in SβG Diluent (Quanterix). 100 μl of SβG was added to each well and the plate was incubated at room temperature with shaking for 20 minutes. The beads were then washed eight times with System Wash Buffer 1. The enzyme-labelled beads were then reconstituted in a dilution buffer (1× sodium chloride-sodium phosphate-EDTA [SSPE] and 1.6% dextran sulfate in DEPC-treated water), transferred onto a new 96-well plate (Quanterix) and loaded onto the HD-1 Analyzer (Quanterix) for analysis. All samples were measured in triplicate unless otherwise noted. Resorufin β-D-galactopyranoside (RGP), Wash Buffer 1, Wash Buffer 2, and Simoa Sealing Oil were purchased from Quanterix and loaded onto the Simoa HD-1 Analyzer based on the manufacturer's instructions. In the HD-1 Analyzer software, the assay was defined based on the “Acute care assay neat 2.0” with an incubation time of 1 cadence.

Direct Detection of miRNA in Human Serum Using Simoa Assays

Healthy human serum samples were purchased from BioReclamationlVT. Sodium dodecyl sulfate (SDS, final w/v 2%) and Proteinase K (final 0.16 U/ml) (New England Biolabs) were added to the serum samples. The serum samples were vortexed and incubated at room temperature for 15 minutes. The serum samples were then heated to 90° C. for two minutes, diluted in hybridization buffer, and spiked with synthetic miRNA to desired concentrations.

Detection of microRNAs in Total RNA Samples using Simoa Assays

Human Lung Total RNA (ThermoFisher, AM7968) was serially diluted 10-fold in hybridization buffer and tested using the three-plex Simoa assay for miR-21, miR-141, and miR-16. To determine concentrations of miRNAs in these samples, a calibration curve for each target miRNA was fit to a 4PL nonlinear regression with a 1/y² weighting factor, and unknown values were interpolated using the Simoa HD-1 Analyzer Software (Quanterix).

Detection of microRNAs in Total RNA using qPCR

The following RT-qPCR reagents were purchased from ThermoFisher: qPCR Taqman assays for miR-21, miR-141, and miR-16 (4440886), TaqMan Universal Master Mix II (4440043), and TaqMan MicroRNA Reverse Transcription Kit (4366596). Reverse transcription of known standards and four dilutions of Human Lung Total RNA, corresponding to 100 ng, 10 ng, 1 ng, and 0.1 ng of total RNA, was performed in triplicate. qPCR was performed based on the manufacturer's instructions using a CFX96 real-time PCR system and CFX Manager Software for data analysis (Biorad).

### Results

Simoa Assay with a Bead-Based Sandwich Protocol

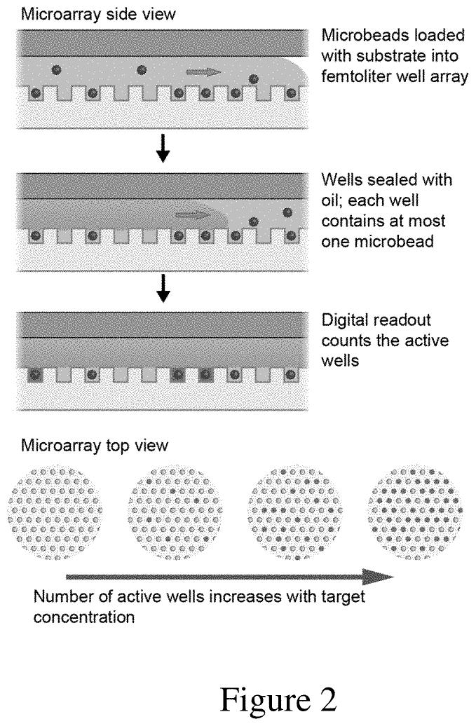

The Simoa assay was applied to miRNA detection by developing a bead-based sandwich protocol as shown in FIG. 1. To increase hybridization specificity, LNA-modified capture and detection probes with sequences complementary to either 11 or 12 bases of the target miRNA were used as shown in Table 1. Before performing the Simoa assay, LNA-modified capture probes specific to a target miRNA were covalently coupled to paramagnetic microbeads. A sample containing the target miRNA was incubated with the capture probe-coupled microbeads and biotinylated detection probes, forming a sandwich complex. The beads were then washed to remove unbound miRNA. The beads labelled with both target miRNA and biotinylated detection probes were then labelled with an enzyme, SβG, via biotin-streptavidin interaction and detected by enzymatic readout in the Simoa platform as shown in FIG. 2. The signal from the assay was measured in units of average enzyme per bead (AEB).

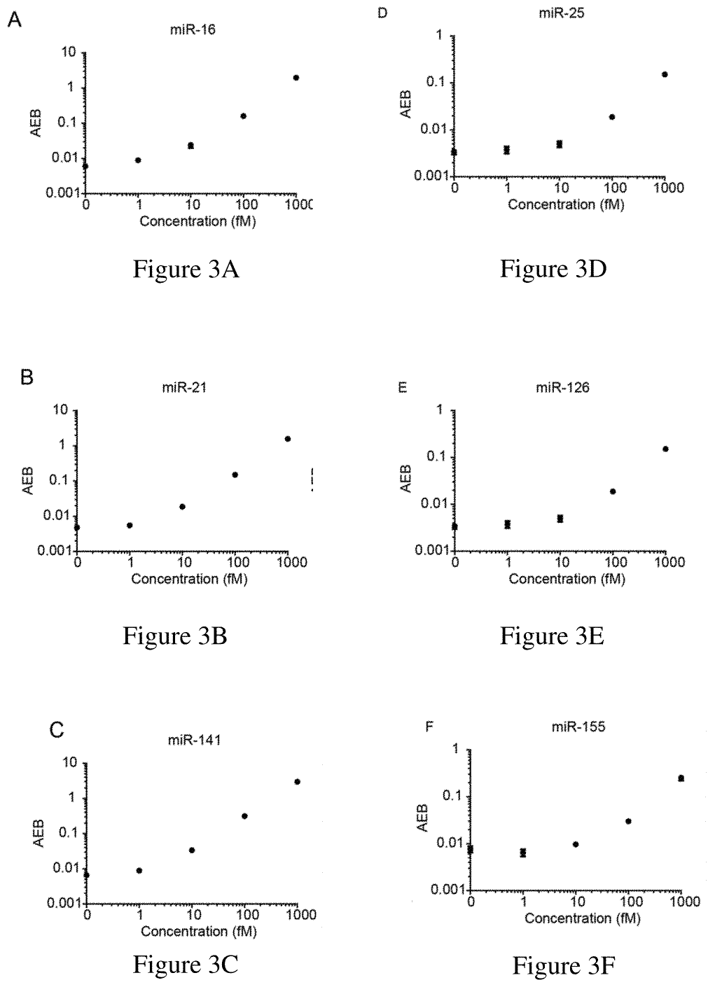

To test the performance of the assay, several target miRNAs were selected that have been previously associated with cancer. These targets include miR-16, miR-21, miR-141, miR-25, miR-126, and miR-155. The melting temperatures of these miRNAs are representative of the general population of miRNAs as described in Example 2. Known concentrations of each target miRNA were then measured using the single-plex assays. As shown in FIG. 3, similar performance for all assays was obtained with limits of detection of below 1 to 30 femtomolar.

Multiplexed Detection of miRNAs

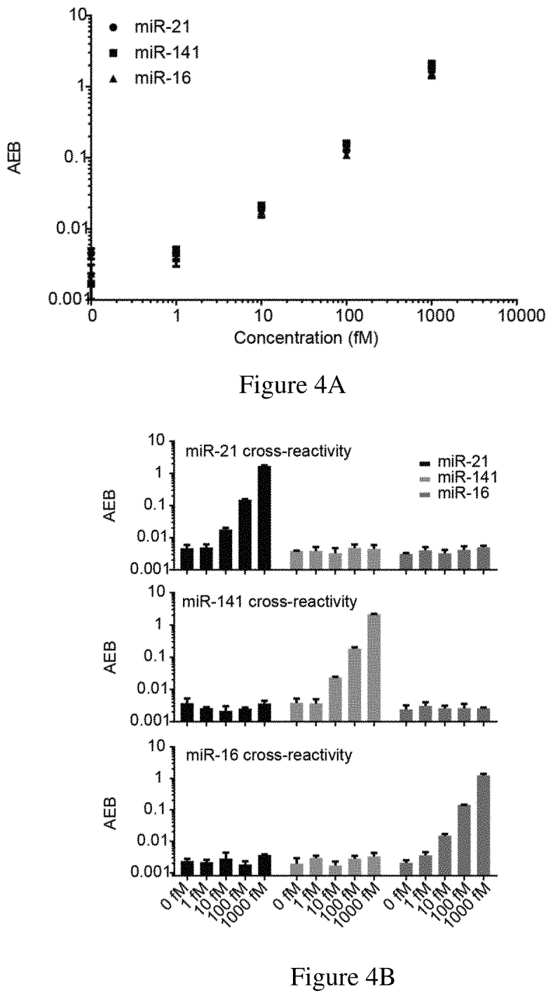

Simultaneous detection of multiple different target miRNAs in a single sample increases throughput and requires less sample volume compared to detection of each target individually. When making multiplexed measurements, the presence of multiple target miRNAs in a sample can potentially introduce cross-reactivity that limits the practical utility of an assay. Three widely-used miRNA biomarkers, miR-16, miR-21, and miR-141, were chosen to test direct detection approach in a multiplex format. To enable multiplexing, paramagnetic beads labelled with different fluorescent dyes were used to produce distinct bead subpopulations. Each subpopulation of beads was then further modified with capture probes for a specific miRNA. After incubation with three specific biotinylated detection probes, three miRNAs were measured simultaneously with an average limit of detection of approximately 10 femtomolar as shown in FIG. 4A, demonstrating that multiplexing does not compromise sensitivity. To assess specificity, the multiplex assay was tested with increasing concentrations of each target miRNA individually. See FIG. 4B. Results showed this multiplex assay demonstrated high specificity for its intended targets, with minimal off-target signals even at high concentrations.

Multiplexed Detection of Homologous miRNAs with a Single Nucleotide Mismatch

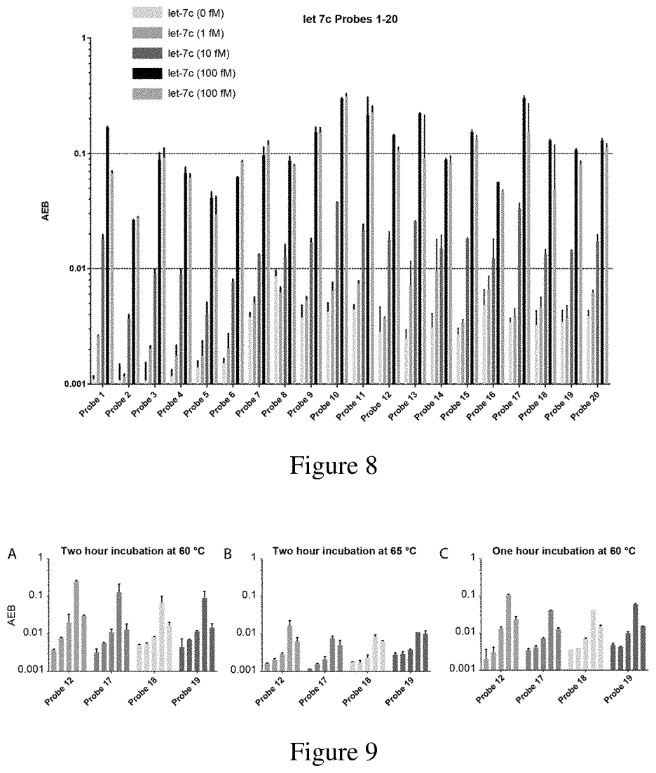

A major challenge in miRNA detection is distinguishing between miRNAs with very similar sequences. The challenge of cross-reactivity becomes especially pronounced when performing multiplexed measurements, as the number of probes and potential off-targets increases. To evaluate the specificity of the Simoa direct detection approach against highly homologous miRNAs, miRNAs that differed by only one or two nucleotides were tested. As representative target miRNAs with highly similar sequences, three members from the human let-7 family: let-7a, let-7b, and let-7c were chosen. 20 different capture probes, specific to let-7c, with varying melting temperatures and number of LNA bases were designed as shown in Table 2. The selectivity of each probe against increasing concentrations (0 fM, 1 fM, 10 fM, and 100 fM) of let-7c was tested and the specificity of these capture probes against 100 fM of let-7b, which differs by a single nucleotide was also tested. FIG. 8 shows the signal from various designs of let-7c capture probes, whose sequences are given in Table 2. Each probe was tested against 0 fM, 1 fM, 10 fM, and 100 fM of let-7c, as well as 100 fM of let-7b. Measurements were obtained in duplicate. This screen was performed at room temperature and four hours of incubation. To identify probes with low cross-reactivity, the on-target to off-target ratio (signal at 100 fM of let-7c over signal of 100 fM of let-7b) was compared. Notably, probe 1, which did not contain any LNA bases, was the least cross-reactive. The signal to noise ratio (signal at 100 fM of let7c over signal of the blank) was then compared and probes 12, 17, 18, and 19 were selected for further optimization. Probes 12, 17, 18, and 19 were selected for further optimization. The effects of incubation time and temperature on assay performance were tested. Each probe was tested against 0 fM, 1 fM, 10 fM, and 100 fM of let-7c, as well as 100 fM of let-7b. FIG. 9 shows the results for (A) incubation performed at 60° C. for two hours, (B) incubation performed at 65° C. for two hours, and (C) incubation performed for one hour at 60° C. For these four probes, it was observed that higher specificity was obtained at 60° C., while the assay signal was substantially lower at 65° C. (closer to the melting temperature of the probes). Probe 12 was chosen for further multiplexed assay development. A clear correlation between the performance of the Simoa assay, the number of LNA residues, and the predicted melting temperature of the probes was not observed.

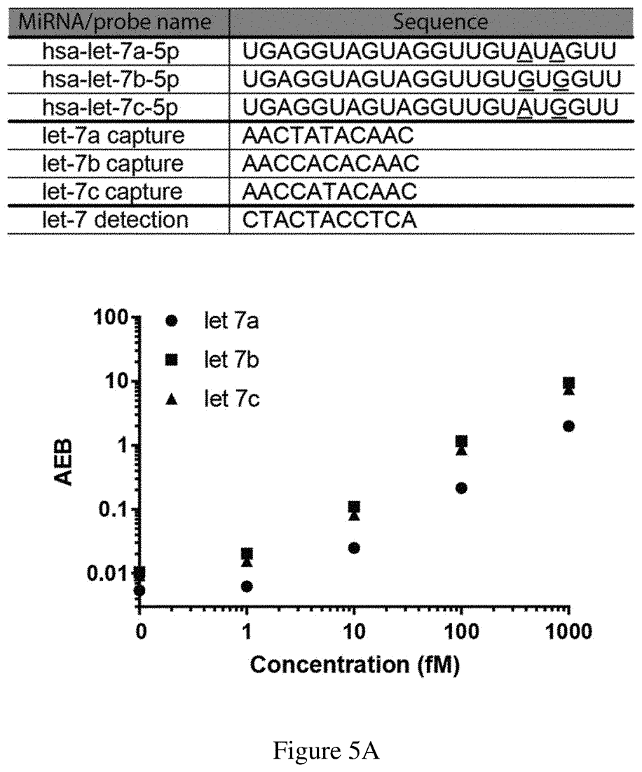

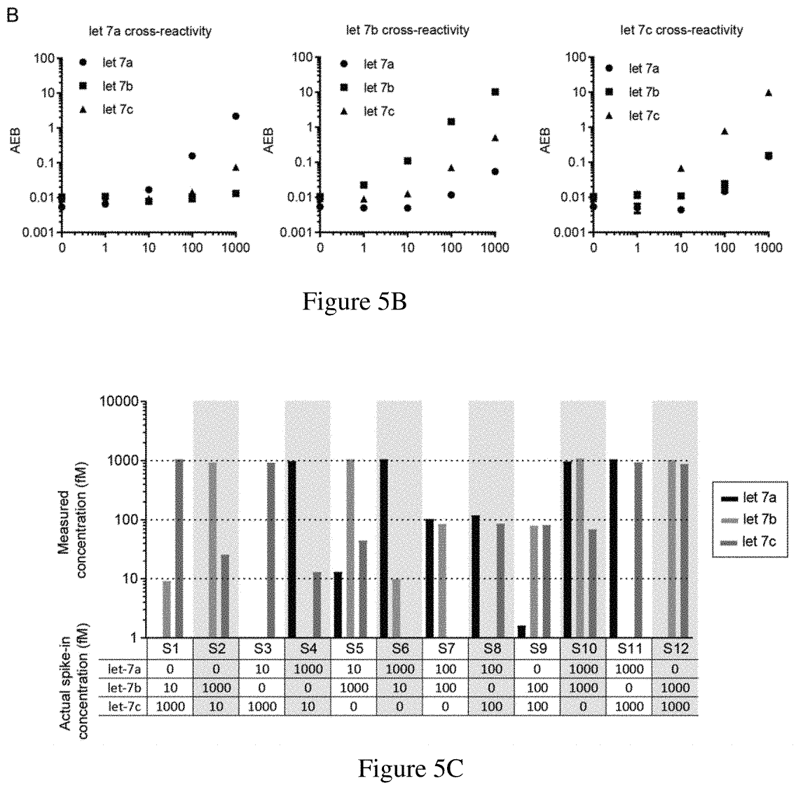

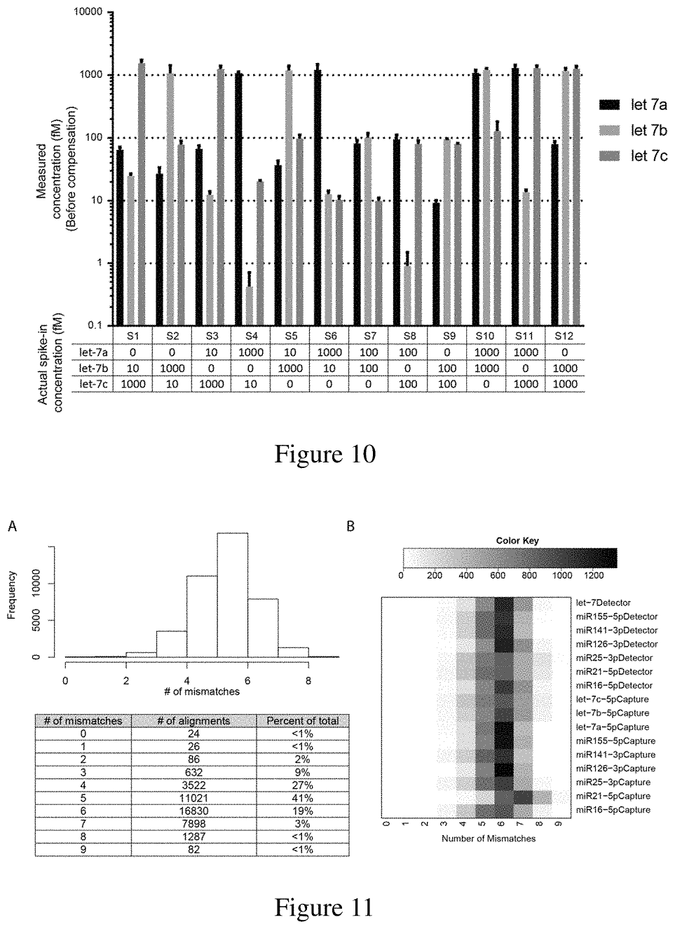

Based on these results, a three-plex Simoa assay to measure let-7a, let-7b, and let-7c simultaneously was developed. See FIG. 5A. For the let-7a and let-7b probes, a probe design was chosen in which the LNA bases were placed in the same positions as the let-7c probe. Varying concentrations of let-7a, let-7b, and let-7c were spiked in, in the absence of the other two targets, to determine the cross-reactivity of each target miRNA against the different capture probes. See FIG. 5B. Mixed samples of varying concentrations of let-7a, let-7b, and let-7c were then tested. See FIG. 5C. The initial measurements of these mixed samples were not accurate due to cross-reactivity as shown in FIG. 10. To compensate for the cross-reactivity of off-target miRNAs, a correction to the signal was applied by fitting the corresponding cross-reactivity curve. The signal at each plex was fit to a linear or four-parameter logistic (4PL) fit. Fit parameters are shown in the Table 3. The concentration of each target miRNA was determined by solving a series of equations. Post-corrected data is shown in FIG. 5C. The fit equations were as follows:

Combined signal for each plex:

Sₐ=fₐₐ(xₐ)+f_{ba}(x_{b})+f_{ca}(x_{c})

S_{c}=f_{ac}(xₐ)+f_{bc}(x_{b})+f_{cc}(x_{c})

Linear fit: f(x)=mx+b

4PL fit:

$$f ( x ) = D + \frac{A - D}{1 + {( \frac{x}{C} )}^{B}}$$

TABLE 3
Fit parameters for multiplex assay.

| | let7a Standard | | let7b Standard | | | let7c Standard | | |
|---|---|---|---|---|---|---|---|---|
| | 7a | 7c | 7a | 7b | 7c | 7a | 7b | 7c |
| A | 0.005545 | 0.00904 | 0.005125 | 0.01107 | 0.009044 | 0.004947 | 0.01112 | 0.00909 |
| B | 1.141 | 1.29 | 5.293 | 1.097 | 1.279 | 5.743 | 4.541 | 1.142 |
| C | 6683781 | 1468 | 142.5 | 1342 | 708.7 | 157.3 | 176.9 | 6485 |
| D | 49913 | 0.1822 | 0.05438 | 24.65 | 0.8271 | 0.1462 | 0.1564 | 92.73 |
| R² | 0.9921 | 0.965 | 0.9638 | 0.9796 | 0.9656 | 0.9273 | 0.9394 | 0.9729 |

| | 7b |
|---|---|
| m | 3.53E−06 |
| b | 0.009619476 |
| R² | 0.6216501 |

The resulting measurements of target miRNAs were in good agreement with the spiked-in concentrations. In samples S5 and S10, measurements of let-7c were substantially higher than the actual spike-in concentrations even after correcting for cross-reactivity. Nevertheless, the assay was able to accurately quantify the concentrations of let-7a, let-7b, and let-7c in most of these samples.

Measuring miRNA Concentrations Spiked into Human Serum

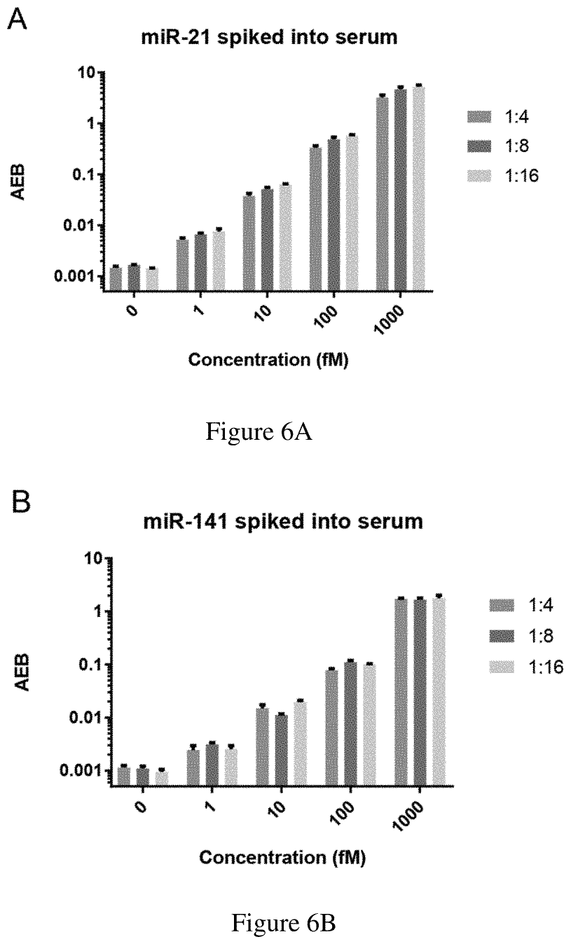

Detection of circulating miRNAs in serum is a promising strategy for minimally-invasive diagnostics. While measurements of miRNAs in serum often involve a preliminary RNA isolation step, the potential for detecting miRNAs directly in serum without RNA isolation was explored. One major challenge for direct detection of miRNAs in serum is the influence of matrix effects that may interfere with the assay. To evaluate the use of the Simoa direct detection assay with serum samples, known concentrations of miR-21 and miR-141 were spiked into healthy human serum. When miRNA was spiked directly into untreated serum, the miRNA was undetectable, presumably due to degradation. However, when the serum was pre-treated with Proteinase K and SDS, followed by heating, the spiked-in miRNAs were detectable, as previously reported. As shown in FIG. 6A-6B, further dilutions of serum in hybridization buffer had a relatively small effect on the measurements. These results suggest that, at least for dilution factors below 1:4, matrix effects from the serum do not interfere substantially with the detection process. Notably, 1 femtomolar of spiked-in miRNA was detectable in the serum matrix.

miRNA Detection in Total RNA

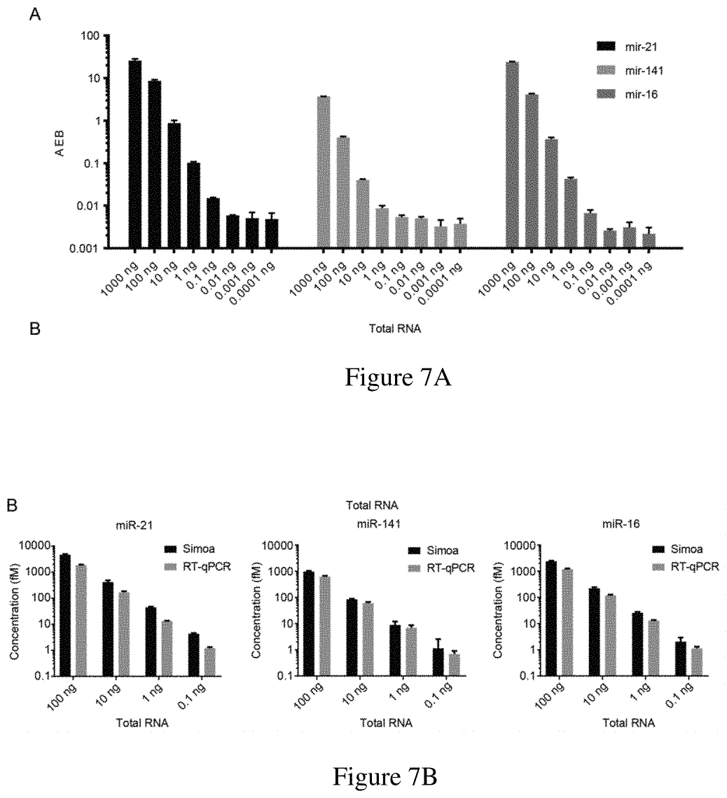

Accurate quantification of miRNAs in total RNA is useful for applications in both fundamental biology and clinical diagnostics. To explore the utility of the Simoa assay for detecting miRNAs in total RNA, a commercially purchased sample of total RNA isolated from cell lysates was tested. Each of these samples was serially diluted ten-fold and miR-21, miR-141, and miR-16 was measured using the three-plex Simoa assay. As shown in FIG. 7A, endogenous miRNAs were detectable and readily quantifiable, exhibiting a linear trend that corresponded to the decreasing concentration of total RNA. The miRNAs were measured over a wide range, which spanned four orders of magnitude, and with a low LOD of approximately 10 femtomolar, corresponding to 0.1 ng of total RNA. As a control, a subset of samples were also tested against the beads from the let-7 multiplex assay to confirm that the signal was not due to non-specific binding (data not shown). To confirm the accuracy of the results, RT-qPCR was used against a set of known standards for use as a calibration curve and a subset of the samples that were previously tested using the Simoa assay. As shown in FIG. 7B, the RT-qPCR results were in good agreement with the Simoa measurements for both relative and absolute quantification.

The emergence of miRNAs as potential biomarkers for cancer and other diseases necessitates new approaches to detect miRNAs with high sensitivity and specificity. RT-qPCR remains the gold standard method for nucleic acid detection, but suffers from target amplification bias, sample loss due to reverse transcription, and lack of multiplexing capabilities. In addition, miRNAs represent particularly challenging targets for RT-qPCR due to their short sequence length, requiring nonstandard primers. Direct detection of miRNAs is a promising alternative to RT-qPCR but it is often difficult to detect low concentrations of miRNAs without target amplification. In this example, a direct detection assay, based on the Simoa technology, was developed, which is capable of extremely high-sensitivity measurements of miRNAs. The method reported here is the first implementation of Simoa for miRNA detection. There are several advantages to this method, including high sensitivity, specificity, minimal processing steps, and multiplexing capabilities.

Additionally, in the Simoa direct detection approach, both capture and detection probes must bind to the target miRNA to produce a signal. Consequently, cross-reactivity from off-target miRNAs was not expected to have substantial effects when using the Simoa approach to measure miRNAs. Some miRNA families, such as the let-7 family, have a high degree of homology; in these cases, the signal arising from cross-hybridization must be accounted for. Using this approach, highly homologous miRNAs that differed by only one or two nucleotides were successfully measured.

The Simoa-based direct detection approach was applied to measurement of miRNAs in serum without a separate isolation step. It was observed that spiked-in miRNA was undetectable in untreated serum; however, when the serum was pre-treated, 1 fM of spiked miRNAs was detectable as shown in FIG. 6. This result is consistent with previous findings that show exogenous miRNAs are undetectable upon addition to serum or blood, while endogenous miRNAs are stable. Detection of three different miRNAs simultaneously in samples containing as little as 0.1 ng total RNA was also successfully demonstrated. When the accuracy of this direct detection approach was compared to the current gold standard tool, RT-qPCR, the results were in good agreement as shown in FIG. 7. Thus, this Simoa direct detection approach can provide highly sensitive, multiplexed, and accurate quantification of miRNAs.

The direct hybridization approach described here does not require pre-labelling, reverse transcription, or amplification steps. The total time required for the assay is about five hours, including about 1.5 hours of “hands on” time. Furthermore, multiplexing capabilities allowed measurements of several miRNAs simultaneously and thus enhance efficiency. The simple and automated nature of this assay suggests that it can easily scale up to higher throughput for routine testing. Additionally, the high sensitivity and specificity of the Simoa direct detection approach make it a promising tool for miRNA detection.

### Example 2

### Design of Probes for Use in Detection of MicroRNAs

This example further describes the probe design in Example 1.

In order to explore the sequence-specificity and generality of the sandwich hybridization approach, the entire population of known human miRNAs, collected in the miRbase database, were considered in terms of two important parameters for the assay: sequence similarity and melting temperature.

### Sequence Similarity

Alignment between Probes Tested in Example 1 and All miRNA Sequences

Distinguishing between homologous miRNAs can be difficult, whether sequence similarity between miRNAs would pose a serious challenge to the specificity of the assay was explored. First, the sequence complementarity between the individual capture and detector probes used in Example 1 and the population of human miRNAs in miRbase were considered. Match scores were calculated by a pairwise alignment of all probes, using a gapless Smith-Waterman algorithm, against a database of all Homo sapiens miRNA sequences. (Sequences were obtained from the “mature.fa” listing of all mature miRNA sequences from the miRbase database.) FIG. 11 shows the distribution of number of mismatches in pairwise alignments between probes used in Example 1 (shown in Table 1) and the broader human miRNA population. In total, 16 probes compared against 2,588 miRNA sequences give 41,408 pairwise alignments. The resulting distribution shows that the probes in Example 1 have low complementarity with off-target miRNA biomarkers, with four or more mismatches for the vast majority of miRNA biomarkers. The heatmap shows the frequency of mismatches for each of the probes used in Example 1.

Alignment Between Putative Probes and All miRNA Sequences

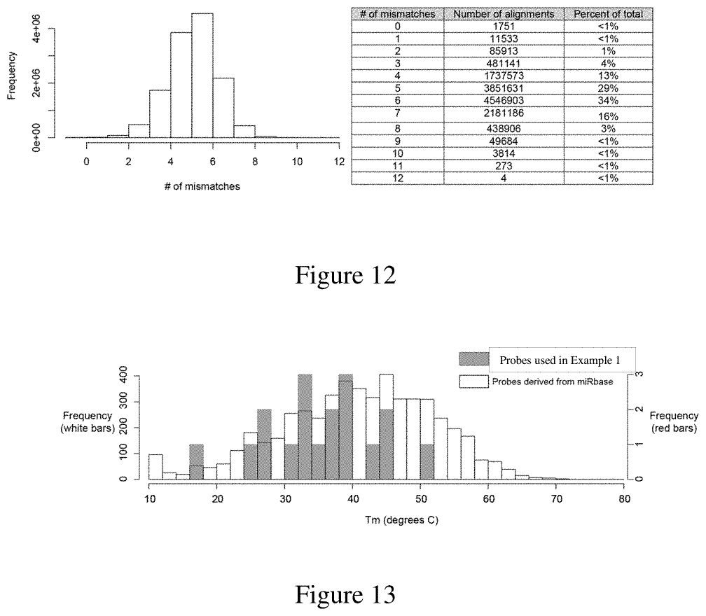

Each human miRNA in miRbase (from the “mature.fa” listing) was divided into two probe-binding regions termed “putative probes”. miRNA with an odd number of nucleotides resulted in one odd-length and one even-length probe. Alignment scores were obtained for each putative probe against each miRNA. Starting from 2,588 miRNA entries, there were 5,176 putative probes, leading to 2,588×5,176=13,395,488 pairwise alignments. Among these alignments, 5,176 correspond to “on-target” matches (where the putative probe was compared against the sequence from which it was derived), which were excluded from FIG. 12.

### Melting Temperature (Tₘ)

Both sensitivity and specificity of hybridization-based assays are strongly dependent on melting temperature. In the sandwich hybridization approach, about half of each miRNA was bound with a probe. If it is assumed that each miRNA sequence is divided in half (rounded to the nearest nucleotide), with one half hybridizing to a capture probe and the other half hybridizing to a detection probe, the Tₘ of each probe-binding region can be estimated. FIG. 13 shows the distribution of calculated melting temperatures for putative probes derived from human mature miRNA. As shown in FIG. 11, each human miRNA in miRbase was divided into two “putative probes”. Melting temperatures were calculated using the nearest neighbor model for RNA-RNA interactions with parameters from Xia, Biochemistry, 37(42):14719 (1998). For these calculations, oligo concentration was assumed to be 1 nM and salt concentration adjustment was made assuming 115 mM Na+. Melting temperatures at or below 10° C. were binned in the resulting histogram. As the resulting Tₘ distribution shows (white bars, left axis), a strategy of splitting each miRNA into two probe-binding regions results in a modal Tₘ of 40-45° C. For reference, the calculated Tₘ's of probes used in Example 1 are overlaid on the histogram (gray bars, right axis).

The estimated melting temperatures of probes used in Example 1 reflect the distribution of melting temperatures in the human miRNA population. As shown in FIG. 13, melting temperatures were calculated using the nearest neighbor (NN) model with parameters from Xia et al., Biochemistry, 37(42):14719 (1998). The Tₘ of probe-binding regions that were tested empirically in this example (total of 18 probes tested) ranged from 17.6 to 51.2° C. This range of melting temperatures covers about 80% of the probe-binding regions in the distribution as shown in FIG. 13.

TABLE 4
Probes and their estimated melting temperature.

| | | | Estimated |
|---|---|---|---|
| miRNA name/probe# | miRbase Acc. # | Sequence | Tₘ (° C.) |
| hsa-miR-16-5p/1 | MIMAT0000069 | UAGCAGCACGU | 45.5 |
| | | (SEQ ID NO: 46) | |
| hsa-miR-16-5p/2 | MIMAT0000069 | AAAUAUUGGCG | 27.8 |
| | | (SEQ ID NO: 47) | |
| hsa-miR-21-5p/1 | MIMAT0000076 | UAGCUUAUCAG | 32.4 |
| | | (SEQ ID NO: 48) | |
| hsa-miR-21-5p/2 | MIMAT0000076 | ACUGAUGUUGA | 33.5 |
| | | (SEQ ID NO: 49) | |
| hsa-miR-25-3p/1 | MIMAT0000081 | CAUUGCACUUG | 34.7 |
| | | (SEQ ID NO: 50) | |
| hsa-miR-25-3p/2 | MIMAT0000081 | UCUCGGUCUGA | 45.4 |
| | | (SEQ ID NO: 51) | |
| hsa-miR-126-3p/1 | MIMAT0000445 | UCGUACCGUGA | 43.8 |
| | | (SEQ ID NO: 52) | |
| hsa-miR-126-3p/2 | MIMAT0000445 | GUAAUAAUGCG | 27.4 |
| | | (SEQ ID NO: 53) | |
| hsa-miR-141-3p/1 | MIMAT0000432 | UAACACUGUCU | 33.9 |
| | | (SEQ ID NO: 54) | |
| hsa-miR-141-3p/2 | MIMAT0000432 | GGUAAAGAUGG | 36.2 |
| | | (SEQ ID NO: 55) | |
| hsa-miR-155-5p/1 | MIMAT0000646 | UUAAUGCUAAU | 17.6 |
| | | (SEQ ID NO: 56) | |
| hsa-miR-155-5p/2 | MIMAT0000646 | CGUGAUAGGGGU | 51.2 |
| | | (SEQ ID NO: 57) | |
| hsa-let-7a-5p/1 | MIMAT0000062 | UGAGGUAGUAG | 39.7 |
| | | (SEQ ID NO: 58) | |
| hsa-let-7a-5p/2 | MIMAT0000062 | GUUGUAUAGUU | 24.5 |
| | | (SEQ ID NO: 59) | |
| hsa-let-7b-5p/1 | MIMAT0000063 | UGAGGUAGUAG | 39.7 |
| | | (SEQ ID NO: 60) | |
| hsa-let-7b-5p/2 | MIMAT0000063 | GUUGUGUGGUU | 37.6 |
| | | (SEQ ID NO: 61) | |
| hsa-let-7c-5p/1 | MIMAT0000064 | UGAGGUAGUAG | 39.7 |
| | | (SEQ ID NO: 62) | |
| hsa-let-7c-5p/2 | MIMAT0000064 | GUUGUAUGGUU | 31.3 |
| | | (SEQ ID NO: 63) | |

This consideration of melting temperatures allowed the probes used in Example 1 to be placed into context, as it provided a basis for comparison with the broader miRNA population. The melting temperature estimates in FIG. 13 and Table 4 were calculated using the nearest neighbor model for RNA-RNA interactions; this calculation assumes no LNA bases, as well as many simplifying assumptions such as solution phase hybridization. In some instances, LNA bases were incorporated to increase the Tₘ of the probe. The use of partial-LNA probes provided an additional degree of freedom that allowed the melting temperature to be normalized, so consistent conditions could be used for multiple miRNA biomarkers. In addition, the use of probes with higher Tm allowed the hybridization temperature to be increased and improved specificity of the hybridization.

The Simoa direct detection approach can be used to measure a wide range of miRNAs. Several considerations must be taken into account when designing the LNA-modified capture and detection probes. Flexibility in probe design is limited due to the short length of miRNAs and thus the complementary sequence of the capture and detection probe pairs is pre-determined by the sequence of the target miRNA. LNA-modified probes can be used to increase specificity and have frequently been used for specific hybridization and multiplexed detection of miRNAs. A general LNA probe design strategy requires consideration of self-complementarity and cross-hybridization with other probes, which must be avoided to reduce the background signal. These considerations are particularly important in multiplexed assays, in which the number of probes and potential cross-reactive off targets increases. Additionally, it is important to ensure that the melting temperature for both capture and detection probes is similar. Due to the short length of miRNAs, it is often not possible to obtain similar melting temperatures for the capture and detection probes. High sensitivity of this Simoa direct detection approach even when the melting temperatures of the capture and detection probes differed by over 30° C. has demonstrated, as exemplified by the miR-155 assay. See Table 4.

Another challenge with miRNA detection is the ability to distinguish between homologous miRNAs. Sequence analysis of the general population of mature human miRNAs against the capture and detection probes used in Example 1 revealed that over 96% of the probes contain three or more mismatches as shown in FIG. 11.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited

1. A method for detecting a target nucleic acid in a sample, comprising:
(a) providing a sample suspected of containing a first target nucleic acid, wherein the first target nucleic acid is about 15-50-nucleotides in length;
(b) incubating the sample with a first capture probe and a first detection probe to form a first complex of the first target nucleic acid, the first capture probe, and the first detection probe; wherein the first capture probe is immobilized on a first support member and the first detection probe is conjugated to a first labeling agent;
(c) washing the first complex to remove unbound first detection probes;
(d) measuring a first signal released, directly or indirectly, from the first labeling agent in the first complex; and
(e) determining presence or a level of the first target nucleic acid in the sample based on the intensity of the first signal obtained in step (d);
wherein the first capture probe and the first detection probe each comprise a nucleotide sequence that is complementary to a first segment of the first target nucleic acid and a second segment of the first target nucleic acid, respectively, and wherein the first segment and second segment of the first target nucleic acid do not overlap.

2. The method of claim 1, wherein the first target nucleic acid is about 18-25-nucleotides in length.

3. The method of claim 1, wherein the first target nucleic acid is an RNA molecule.

4. The method of claim 3, wherein the first target nucleic acid is a mature microRNA.

5. The method of claim 1, wherein the first segment and the second segment of the first target nucleic acid differ in length by less than or equal to about 5 nucleotides.

6. The method of claim 1, wherein the first capture probe, the first detection probe, or both comprise one or more locked nucleic acids (LNAs).

7. The method of claim 1, wherein the first capture probe and the first detection probe collectively are complementary to the whole length of the first target nucleic acid.

8. The method of claim 1, wherein the first target nucleic acid is not enriched or amplified prior to step (a).

9. The method of claim 1, wherein the incubating step (b) is performed at a temperature between about 20° C. and about 65° C.

10.-14. (canceled)

15. The method of claim 1, wherein the first support member is a magnetic bead.

16. The method of claim 1, wherein the first labeling agent is biotin.

17. The method of claim 16, wherein the measuring step (d) is performed using an enzyme conjugated to streptavidin.

18. The method of claim 1, wherein the measuring step (d) is performed by a single molecule array assay.

19.-23. (canceled)

24. The method of claim 1, wherein the first capture probe, the first detection probe, or both have a melting temperature ranging from about 30° C. and about 90° C.

25. The method of claim 1, wherein the melting temperature of the first capture probe differs from that of the first detection probe by up to about 40° C.

26. The method of claim 1, wherein the sample is suspected of containing a second target nucleic acid, which is about 15-50-nucleotides in length;
wherein in step (b), the sample is further incubated with a second capture probe and a second detection probe to form a second complex of the second target nucleic acid, the second capture probe, and the second detection probe; the second capture probe being immobilized on a second support member and comprising a nucleotide sequence complementary to a first segment of the second nucleic acid and the second detection probe being conjugated to a second labeling agent and comprising a nucleotide sequence complementary to a second segment of the second target nucleic acid, which does not overlap with the first segment; and
wherein the method further comprises measuring a second signal released, directly or indirectly, from the second labeling agent in the second complex; and determining presence or a level of the second target nucleic acid in the sample based on the intensity of the second signal.

27. The method of claim 26, wherein the sample is suspected of containing a third target nucleic acid, which is about 15-50-nucleotides in length;
wherein in step (b), the sample is further incubated with a third capture probe and a third detection probe to form a third complex of the third target nucleic acid, the third capture probe, and the third detection probe; the third capture probe being immobilized on a third support member and comprising a nucleotide sequence complementary to a first segment of the third nucleic acid and the third detection probe being conjugated to a third labeling agent and comprising a nucleotide sequence complementary to a second segment of the third target nucleic acid, which does not overlap with the first segment; and
wherein the method further comprises measuring a third signal released, directly or indirectly, from the third labeling agent in the third complex; and determining presence or a level of the third target nucleic acid in the sample based on the intensity of the third signal.

28.-33. (canceled)

34. A multiplex assay for detecting multiple short target nucleic acids, comprising:
(i) providing a sample suspected of containing multiple target short nucleic acids, each of which is about 15-50-nucleotides in length;
(ii) providing multiple sets of probes, each of which includes a capture probe immobilized on a support member and a detection probe conjugated to a labeling agent, the capture probe and the detection probe being complementary to different portions of a target short nucleic acid; wherein the multiple sets of probes are for detection of different target short nucleic acids;
(iii) incubating the sample with the multiple sets of probes to form multiple complexes each containing a target short nucleic acid and a set of probes;
(iv) washing the multiple complexes to remove unbound detection probes;
(v) measuring signals released, directly or indirectly, from the labeling agents in the complexes; and
(vi) determining presence or levels of the multiple short target nucleic acids based on the intensity of the signals detected in step (v).

35.-55. (canceled)

56. A kit for detecting a target nucleic acid, comprising:
(i) a capture probe immobilized on a support member; and
(ii) a detection probe conjugated to a labelling agent,
wherein the target nucleic acid is about 15-50-nucleotides in length, wherein the capture probe and the detection probe each comprise a nucleotide sequence that is complementary to a first segment of the target nucleic acid and a second segment of the target nucleic acid, respectively, and wherein the first segment and second segment of the nucleic acid do not overlap.

57.-65. (canceled)

66. A kit for detecting multiple short target nucleic acids, comprising multiple sets of probes, each of which comprises:
(i) a capture probe immobilized on a support member; and
(ii) a detection probe conjugated to a labelling agent;
wherein each of the short target nucleic acids is about 15-50-nucleotides in length;
wherein in each probe set, the capture probe and the detection probe are complementary to different portions of a short target nucleic acid; and
wherein the multiple sets of probes are for detection of different target nucleic acids.

67.-76. (canceled)


SEQUENCE LISTING (70 sequences)

SEQ ID NO: 1 - RNA, 22 nt, Artificial Sequence
  Synthetic polynucleotide
uagcagcacguaaauauuggcg

SEQ ID NO: 2 - RNA, 22 nt, Artificial Sequence
  Synthetic polynucleotide
uagcuuaucagacugauguuga

SEQ ID NO: 3 - RNA, 22 nt, Artificial Sequence
  Synthetic polynucleotide
cauugcacuugucucggucuga

SEQ ID NO: 4 - RNA, 22 nt, Artificial Sequence
  Synthetic polynucleotide
ucguaccgugaguaauaaugcg

SEQ ID NO: 5 - RNA, 22 nt, Artificial Sequence
  Synthetic polynucleotide
uaacacugucugguaaagaugg

SEQ ID NO: 6 - RNA, 23 nt, Artificial Sequence
  Synthetic polynucleotide
uuaaugcuaaucgugauaggggu

SEQ ID NO: 7 - RNA, 22 nt, Artificial Sequence
  Synthetic polynucleotide
ugagguaguagguuguauaguu

SEQ ID NO: 8 - RNA, 22 nt, Artificial Sequence
  Synthetic polynucleotide
ugagguaguagguugugugguu

SEQ ID NO: 9 - RNA, 22 nt, Artificial Sequence
  Synthetic polynucleotide
ugagguaguagguuguaugguu

SEQ ID NO: 10 - DNA, 17 bp, Artificial Sequence
  Synthetic polynucleotide
  misc_feature (1)..(1): Modified by 5′ Amino Modifier C12
  misc_feature (9)..(9): Locked nucleic acid
  misc_feature (11)..(11): Locked nucleic acid
  misc_feature (13)..(13): Locked nucleic acid
  misc_feature (15)..(15): Locked nucleic acid
  misc_feature (17)..(17): Locked nucleic acid
ttttttcgccaatattt

SEQ ID NO: 11 - DNA, 17 bp, Artificial Sequence
  Synthetic polynucleotide
  misc_feature (1)..(1): Modified by 5′ Amino Modifier C12
  misc_feature (7)..(8): Locked nucleic acid
  misc_feature (10)..(11): Locked nucleic acid
  misc_feature (17)..(17): Locked nucleic acid
tttttttcaacatcagt

SEQ ID NO: 12 - DNA, 17 bp, Artificial Sequence
  Synthetic polynucleotide
  misc_feature (1)..(1): Modified by 5′ Amino Modifier C12
  misc_feature (8)..(8): Locked nucleic acid
  misc_feature (12)..(12): Locked nucleic acid
  misc_feature (16)..(16): Locked nucleic acid
tttttttcagaccgaga

SEQ ID NO: 13 - DNA, 17 bp, Artificial Sequence
  Synthetic polynucleotide
  misc_feature (1)..(1): Modified by 5′ Amino Modifier C12
  misc_feature (9)..(9): Locked nucleic acid
  misc_feature (11)..(12): Locked nucleic acid
  misc_feature (14)..(15): Locked nucleic acid
ttttttcgcattattac

SEQ ID NO: 14 - DNA, 17 bp, Artificial Sequence
  Synthetic polynucleotide
  misc_feature (1)..(1): Modified by 5′ Amino Modifier C12
  misc_feature (8)..(8): Locked nucleic acid
  misc_feature (13)..(13): Locked nucleic acid
  misc_feature (16)..(17): Locked nucleic acid
ttttttccatctttacc

SEQ ID NO: 15 - DNA, 17 bp, Artificial Sequence
  Synthetic polynucleotide
  misc_feature (1)..(1): Modified by 5′ Amino Modifier C12
  misc_feature (8)..(9): Locked nucleic acid
  misc_feature (17)..(17): Locked nucleic acid
ttttttacccctatcac

SEQ ID NO: 16 - DNA, 17 bp, Artificial Sequence
  Synthetic polynucleotide
  misc_feature (1)..(1): Modified by 5′ Amino Modifier C12
  misc_feature (8)..(8): Locked nucleic acid
  misc_feature (10)..(10): Locked nucleic acid
  misc_feature (12)..(12): Locked nucleic acid
  misc_feature (14)..(14): Locked nucleic acid
  misc_feature (16)..(16): Locked nucleic acid
ttttttaactatacaac

SEQ ID NO: 17 - DNA, 17 bp, Artificial Sequence
  Synthetic polynucleotide
  misc_feature (1)..(1): Modified by 5′ Amino Modifier C12
  misc_feature (8)..(8): Locked nucleic acid
  misc_feature (10)..(10): Locked nucleic acid
  misc_feature (12)..(12): Locked nucleic acid
  misc_feature (14)..(14): Locked nucleic acid
  misc_feature (16)..(16): Locked nucleic acid
ttttttaaccacacaac

SEQ ID NO: 18 - DNA, 17 bp, Artificial Sequence
  Synthetic polynucleotide
  misc_feature (1)..(1): Modified by 5′ Amino Modifier C12
  misc_feature (8)..(8): Locked nucleic acid
  misc_feature (10)..(10): Locked nucleic acid
  misc_feature (12)..(12): Locked nucleic acid
  misc_feature (14)..(14): Locked nucleic acid
  misc_feature (16)..(16): Locked nucleic acid
ttttttaaccatacaac

SEQ ID NO: 19 - DNA, 11 bp, Artificial Sequence
  Synthetic polynucleotide
  misc_feature (2)..(2): Locked nucleic acid
  misc_feature (10)..(11): Locked nucleic acid
acgtgctgcta

SEQ ID NO: 20 - DNA, 11 bp, Artificial Sequence
  Synthetic polynucleotide
  misc_feature (2)..(2): Locked nucleic acid
  misc_feature (6)..(7): Locked nucleic acid
  misc_feature (9)..(11): Locked nucleic acid
ctgataagcta

SEQ ID NO: 21 - DNA, 11 bp, Artificial Sequence
  Synthetic polynucleotide
  misc_feature (1)..(1): Locked nucleic acid
  misc_feature (3)..(5): Locked nucleic acid
  misc_feature (9)..(11): Locked nucleic acid
caagtgcaatg

SEQ ID NO: 22 - DNA, 11 bp, Artificial Sequence
  Synthetic polynucleotide
  misc_feature (1)..(1): Locked nucleic acid
  misc_feature (4)..(4): Locked nucleic acid
  misc_feature (9)..(9): Locked nucleic acid
  misc_feature (11)..(11): Locked nucleic acid
tcacggtacga

SEQ ID NO: 23 - DNA, 11 bp, Artificial Sequence
  Synthetic polynucleotide
  misc_feature (1)..(2): Locked nucleic acid
  misc_feature (4)..(4): Locked nucleic acid
  misc_feature (6)..(6): Locked nucleic acid
  misc_feature (9)..(9): Locked nucleic acid
  misc_feature (11)..(11): Locked nucleic acid
agacagtgtta

SEQ ID NO: 24 - DNA, 12 bp, Artificial Sequence
  Synthetic polynucleotide
  misc_feature (1)..(2): Locked nucleic acid
  misc_feature (4)..(4): Locked nucleic acid
  misc_feature (7)..(7): Locked nucleic acid
  misc_feature (9)..(10): Locked nucleic acid
  misc_feature (12)..(12): Locked nucleic acid
gattagcattaa

SEQ ID NO: 25 - DNA, 11 bp, Artificial Sequence
  Synthetic polynucleotide
  misc_feature (2)..(2): Locked nucleic acid
  misc_feature (4)..(4): Locked nucleic acid
  misc_feature (6)..(6): Locked nucleic acid
  misc_feature (8)..(8): Locked nucleic acid
  misc_feature (10)..(11): Locked nucleic acid
ctactacctca

SEQ ID NO: 26 - DNA, 17 bp, Artificial Sequence
  Synthetic polynucleotide
  misc_feature (1)..(1): Modified by 5′ Amino Modifier C12
ttttttaaccatacaac

SEQ ID NO: 27 - DNA, 17 bp, Artificial Sequence
  Synthetic polynucleotide
  misc_feature (1)..(1): Modified by 5′ Amino Modifier C12
  misc_feature (7)..(7): Locked nucleic acid
  misc_feature (9)..(9): Locked nucleic acid
  misc_feature (11)..(11): Locked nucleic acid
  misc_feature (13)..(13): Locked nucleic acid
  misc_feature (15)..(15): Locked nucleic acid
  misc_feature (17)..(17): Locked nucleic acid
ttttttaaccatacaac

SEQ ID NO: 28 - DNA, 17 bp, Artificial Sequence
  Synthetic polynucleotide
  misc_feature (1)..(1): Modified by 5′ Amino Modifier C12
  misc_feature (9)..(10): Locked nucleic acid
  misc_feature (14)..(14): Locked nucleic acid
  misc_feature (17)..(17): Locked nucleic acid
ttttttaaccatacaac

SEQ ID NO: 29 - DNA, 17 bp, Artificial Sequence
  Synthetic polynucleotide
  misc_feature (1)..(1): Modified by 5′ Amino Modifier C12
  misc_feature (11)..(13): Locked nucleic acid
ttttttaaccatacaac

SEQ ID NO: 30 - DNA, 17 bp, Artificial Sequence
  Synthetic polynucleotide
  misc_feature (1)..(1): Modified by 5′ Amino Modifier C12
  misc_feature (7)..(9): Locked nucleic acid
  misc_feature (12)..(12): Locked nucleic acid
  misc_feature (16)..(17): Locked nucleic acid
ttttttaaccatacaac

SEQ ID NO: 31 - DNA, 17 bp, Artificial Sequence
  Synthetic polynucleotide
  misc_feature (1)..(1): Modified by 5′ Amino Modifier C12
  misc_feature (7)..(8): Locked nucleic acid
  misc_feature (12)..(12): Locked nucleic acid
  misc_feature (14)..(14): Locked nucleic acid
  misc_feature (17)..(17): Locked nucleic acid
ttttttaaccatacaac

SEQ ID NO: 32 - DNA, 17 bp, Artificial Sequence
  Synthetic polynucleotide
  misc_feature (1)..(1): Modified by 5′ Amino Modifier C12
  misc_feature (9)..(9): Locked nucleic acid
  misc_feature (12)..(12): Locked nucleic acid
  misc_feature (15)..(15): Locked nucleic acid
ttttttaaccatacaac

SEQ ID NO: 33 - DNA, 17 bp, Artificial Sequence
  Synthetic polynucleotide
  misc_feature (1)..(1): Modified by 5′ Amino Modifier C12
  misc_feature (9)..(9): Locked nucleic acid
  misc_feature (12)..(12): Locked nucleic acid
  misc_feature (15)..(15): Locked nucleic acid
  misc_feature (17)..(17): Locked nucleic acid
ttttttaaccatacaac

SEQ ID NO: 34 - DNA, 17 bp, Artificial Sequence
  Synthetic polynucleotide
  misc_feature (1)..(1): Modified by 5′ Amino Modifier C12
  misc_feature (9)..(10): Locked nucleic acid
  misc_feature (12)..(12): Locked nucleic acid
  misc_feature (14)..(14): Locked nucleic acid
  misc_feature (17)..(17): Locked nucleic acid
ttttttaaccatacaac

SEQ ID NO: 35 - DNA, 17 bp, Artificial Sequence
  Synthetic polynucleotide
  misc_feature (1)..(1): Modified by 5′ Amino Modifier C12
  misc_feature (7)..(8): Locked nucleic acid
  misc_feature (10)..(12): Locked nucleic acid
  misc_feature (14)..(15): Locked nucleic acid
  misc_feature (17)..(17): Locked nucleic acid
ttttttaaccatacaac

SEQ ID NO: 36 - DNA, 17 bp, Artificial Sequence
  Synthetic polynucleotide
  misc_feature (1)..(1): Modified by 5′ Amino Modifier C12
  misc_feature (7)..(7): Locked nucleic acid
  misc_feature (9)..(10): Locked nucleic acid
  misc_feature (12)..(14): Locked nucleic acid
  misc_feature (17)..(17): Locked nucleic acid
ttttttaaccatacaac

SEQ ID NO: 37 - DNA, 17 bp, Artificial Sequence
  Synthetic polynucleotide
  misc_feature (1)..(1): Modified by 5′ Amino Modifier C12
  misc_feature (8)..(8): Locked nucleic acid
  misc_feature (10)..(10): Locked nucleic acid
  misc_feature (12)..(12): Locked nucleic acid
  misc_feature (14)..(14): Locked nucleic acid
  misc_feature (16)..(16): Locked nucleic acid
ttttttaaccatacaac

SEQ ID NO: 38 - DNA, 17 bp, Artificial Sequence
  Synthetic polynucleotide
  misc_feature (1)..(1): Modified by 5′ Amino Modifier C12
  misc_feature (8)..(8): Locked nucleic acid
  misc_feature (10)..(10): Locked nucleic acid
  misc_feature (13)..(14): Locked nucleic acid
  misc_feature (16)..(16): Locked nucleic acid
ttttttaaccatacaac

SEQ ID NO: 39 - DNA, 17 bp, Artificial Sequence
  Synthetic polynucleotide
  misc_feature (1)..(1): Modified by 5′ Amino Modifier C12
  misc_feature (8)..(8): Locked nucleic acid
  misc_feature (10)..(11): Locked nucleic acid
  misc_feature (14)..(14): Locked nucleic acid
  misc_feature (16)..(16): Locked nucleic acid
ttttttaaccatacaac

SEQ ID NO: 40 - DNA, 17 bp, Artificial Sequence
  Synthetic polynucleotide
  misc_feature (1)..(1): Modified by 5′ Amino Modifier C12
  misc_feature (8)..(9): Locked nucleic acid
  misc_feature (11)..(11): Locked nucleic acid
  misc_feature (13)..(13): Locked nucleic acid
  misc_feature (16)..(16): Locked nucleic acid
ttttttaaccatacaac

SEQ ID NO: 41 - DNA, 17 bp, Artificial Sequence
  Synthetic polynucleotide
  misc_feature (1)..(1): Modified by 5′ Amino Modifier C12
  misc_feature (8)..(11): Locked nucleic acid
  misc_feature (14)..(14): Locked nucleic acid
ttttttaaccatacaac

SEQ ID NO: 42 - DNA, 17 bp, Artificial Sequence
  Synthetic polynucleotide
  misc_feature (1)..(1): Modified by 5′ Amino Modifier C12
  misc_feature (8)..(9): Locked nucleic acid
  misc_feature (11)..(11): Locked nucleic acid
  misc_feature (13)..(14): Locked nucleic acid
ttttttaaccatacaac

SEQ ID NO: 43 - DNA, 17 bp, Artificial Sequence
  Synthetic polynucleotide
  misc_feature (1)..(1): Modified by 5′ Amino Modifier C12
  misc_feature (8)..(10): Locked nucleic acid
  misc_feature (14)..(14): Locked nucleic acid
  misc_feature (16)..(16): Locked nucleic acid
ttttttaaccatacaac

SEQ ID NO: 44 - DNA, 17 bp, Artificial Sequence
  Synthetic polynucleotide
  misc_feature (1)..(1): Modified by 5′ Amino Modifier C12
  misc_feature (9)..(9): Locked nucleic acid
  misc_feature (11)..(11): Locked nucleic acid
  misc_feature (13)..(14): Locked nucleic acid
  misc_feature (16)..(16): Locked nucleic acid
ttttttaaccatacaac

SEQ ID NO: 45 - DNA, 17 bp, Artificial Sequence
  Synthetic polynucleotide
  misc_feature (1)..(1): Modified by 5′ Amino Modifier C12
  misc_feature (10)..(11): Locked nucleic acid
  misc_feature (13)..(14): Locked nucleic acid
  misc_feature (16)..(16): Locked nucleic acid
ttttttaaccatacaac

SEQ ID NO: 46 - RNA, 11 nt, Artificial Sequence
  Synthetic polynucleotide
uagcagcacgu

SEQ ID NO: 47 - RNA, 11 nt, Artificial Sequence
  Synthetic polynucleotide
aaauauuggcg

SEQ ID NO: 48 - RNA, 11 nt, Artificial Sequence
  Synthetic polynucleotide
uagcuuaucag

SEQ ID NO: 49 - RNA, 11 nt, Artificial Sequence
  Synthetic polynucleotide
acugauguuga

SEQ ID NO: 50 - RNA, 11 nt, Artificial Sequence
  Synthetic polynucleotide
cauugcacuug

SEQ ID NO: 51 - RNA, 11 nt, Artificial Sequence
  Synthetic polynucleotide
ucucggucuga

SEQ ID NO: 52 - RNA, 11 nt, Artificial Sequence
  Synthetic polynucleotide
ucguaccguga

SEQ ID NO: 53 - RNA, 11 nt, Artificial Sequence
  Synthetic polynucleotide
guaauaaugcg

SEQ ID NO: 54 - RNA, 11 nt, Artificial Sequence
  Synthetic polynucleotide
uaacacugucu

SEQ ID NO: 55 - RNA, 11 nt, Artificial Sequence
  Synthetic polynucleotide
gguaaagaugg

SEQ ID NO: 56 - RNA, 11 nt, Artificial Sequence
  Synthetic polynucleotide
uuaaugcuaau

SEQ ID NO: 57 - RNA, 12 nt, Artificial Sequence
  Synthetic polynucleotide
cgugauaggggu

SEQ ID NO: 58 - RNA, 11 nt, Artificial Sequence
  Synthetic polynucleotide
ugagguaguag

SEQ ID NO: 59 - RNA, 11 nt, Artificial Sequence
  Synthetic polynucleotide
guuguauaguu

SEQ ID NO: 60 - RNA, 11 nt, Artificial Sequence
  Synthetic polynucleotide
ugagguaguag

SEQ ID NO: 61 - RNA, 11 nt, Artificial Sequence
  Synthetic polynucleotide
guugugugguu

SEQ ID NO: 62 - RNA, 11 nt, Artificial Sequence
  Synthetic polynucleotide
ugagguaguag

SEQ ID NO: 63 - RNA, 11 nt, Artificial Sequence
  Synthetic polynucleotide
guuguaugguu

SEQ ID NO: 64 - RNA, 22 nt, Artificial Sequence
  Synthetic polynucleotide
ugagguaguagguuguauaguu

SEQ ID NO: 65 - RNA, 22 nt, Artificial Sequence
  Synthetic polynucleotide
ugagguaguagguugugugguu

SEQ ID NO: 66 - RNA, 22 nt, Artificial Sequence
  Synthetic polynucleotide
ugagguaguagguuguaugguu

SEQ ID NO: 67 - DNA, 11 bp, Artificial Sequence
  Synthetic polynucleotide
aactatacaac

SEQ ID NO: 68 - DNA, 11 bp, Artificial Sequence
  Synthetic polynucleotide
aaccacacaac

SEQ ID NO: 69 - DNA, 11 bp, Artificial Sequence
  Synthetic polynucleotide
aaccatacaac

SEQ ID NO: 70 - DNA, 11 bp, Artificial Sequence
  Synthetic polynucleotide
ctactacctca